(19) United States
(12) Patent Application Publication
(10) Pub. No.: US 2004/0077075 A1
(43) Pub. Date: Apr. 22, 2004

(54) Microfermentors for rapid screening and analysis of biochemical processes

(75) Inventors: Klavs F. Jensen, Lexington, MA (US); Paul Laibinis, Houston, TX (US); Rajeev J. Ram, Boston, MA (US); Anthony J. Sinskey, Boston, MA (US); Nicolas Szita, Somerville, MA (US); Andrea Zanzotto, Somerville, MA (US); Martin A. Schmidt, Reading, MA (US)

Correspondence Address: Choate, Hall & Stewart, Exchange Place, 53 State Street, Boston, MA, 02109, US

(73) Assignee: Massachusetts Institute of Technology

(21) Appl. No.: 10/427,373

(22) Filed: May 1, 2003

Related U.S. Application Data
(60) Provisional application No. 60/376,711, filed on May 1, 2002.

(51) Int. Cl. C12M 3/00

(52) U.S. Cl. 435/297.2; 435/297.5; 435/305.3

(57) ABSTRACT

The present invention provides a variety of microscale bioreactors (microfermentors) and microscale bioreactor arrays for use in culturing cells. The microfermentors include a vessel for culturing cells and means for providing oxygen to the interior of the vessel at a concentration sufficient to support cell growth, e.g., growth of bacterial cells. Depending on the embodiment, the microfermentor vessel may have various interior volumes less than approximately 1 ml. The microfermentors may include an aeration membrane and optionally a variety of sensing devices. The invention further provides a chamber to contain the microfermentors and microfermentor arrays and to provide environmental control. Certain of the microfermentors include a second chamber that may be used, e.g., to provide oxygen, nutrients, pH control, etc., to the culture vessel and/or to remove metabolites, etc. Various methods of using the microfermentors, e.g., to select optimum cell strains or bioprocess parameters are provided.

### CROSS-REFERENCE TO RELATED APPLICATION

This application claims priority to U.S. Provisional Patent Application No. 60/376,711, filed May 1, 2002, which is herein incorporated by reference.

### BACKGROUND OF THE INVENTION

A critical driving force behind research in bioprocess science and engineering continues to be the demand for fast and accurate analytical information that can be used, for example, to evaluate the interactions between biological systems and bioprocess operations. One significant challenge is to carry out large numbers of experiments rapidly and efficiently. This issue is of particular importance since many of the advances in molecular biology now lead to large numbers of potential biological systems that contain evolved biocatalysts, new pathway designs, and a variety of unique biological organisms from diverse sources.

Bioprocess development techniques have been unable to keep pace with the current rate of discovery and genetic manipulation in biological systems. Of the hundreds of thousands of genetic and process permutations that can now be designed, only a small fraction can be tested using standard bioprocess practices. Bench-scale bioreactors, with typical volumes of between 2 and 10 liters, are limiting for a number of reasons including the time required to obtain sufficient data for a biological system, the effort required to obtain the data, and the high cost of these systems. Currently the smallest bioreactors that are available commercially have working volumes of approximately 0.5 liters (Sixfors, Appropriate Technical Resources) and allow six parallel fermentations to be carried out.

There exists a need for a platform that allows rapid testing, process development, and optimization to be carried out through parallel fermentations. In particular, there exists a need for microscale bioreactor systems that allow multiple experiments to be performed in parallel without an accompanying increase in cost. In addition, there exists a need for microscale bioreactor systems wherein experimental conditions and results obtained in the microscale bioreactor may be translated into predictable large-scale bioprocess operations.

### SUMMARY OF THE INVENTION

The present invention encompasses the recognition that the ability to perform cell culture, e.g., for testing, strain optimization, bioprocess parameter optimization, etc., in bioreactors with small volumes offers significant advantages as compared with fermentations performed in traditional production scale or bench scale fermentors. Accordingly, the invention provides a variety of microscale bioreactors (microfermentors), microscale bioreactor arrays, and associated apparatus as well as methods for use thereof.

In one aspect, the invention provides a microscale bioreactor (microfermentor) comprising a vessel having an interior volume of less than 200 microliters and means for providing oxygen to the vessel at a concentration sufficient to support cell growth. Optionally, the microfermentor includes at least one channel extending from and in communication with the vessel and/or means for introducing a component into the vessel or removing a sample from the vessel via a channel. According to certain embodiments of the invention the means for providing oxygen comprises an aeration membrane, wherein oxygen diffuses through the membrane into the vessel. The membrane may comprise, for example, a fluoropolymer or a silicone.

In another aspect, the invention provides microscale bioreactors as described above and having means for quantification of biomass, e.g., by measuring the optical density of the culture medium, by measuring the concentration of a cell metabolite, etc. Optionally, the microscale bioreactors may include means for measuring dissolved oxygen within the culture vessel, and/or means for measuring at least one other parameter, which may be, e.g., temperature, pH, carbon dioxide concentration, carbon source concentration, concentration of an ionic species, and concentration of a cellular metabolite.

According to certain embodiments of the invention the means for measuring biomass and/or a bioprocess parameter comprises an optical sensor, e.g., an optical chemical sensor. In certain embodiments of the invention a waveguide sensor is used. According to certain embodiments of the invention Raman spectroscopy is used to measure one or more bioprocess parameters, e.g., concentrations of various organic compounds present in the medium.

In certain aspects of the invention the microscale bioreactors include means for controlling the temperature and/or pH in the culture vessel. The microscale bioreactor systems of the invention may also include means for delivering nutrients and/or for removing a cell product from the culture vessel.

In another aspect, the invention provides two-vessel microscale bioreactors that comprise a first vessel having an interior volume of 1 ml or less for culturing cells and a second vessel separated from the first vessel at least in part by a membrane permeable to oxygen and carbon dioxide. In certain embodiments of the invention the membrane is permeable to cell products and/or nutrients but not permeable to cells. These microscale bioreactor systems may further include means for flowing a liquid or gas through the second vessel.

In another aspect, the invention provides a chamber sufficiently large to accommodate the microscale bioreactor or microscale bioreactor array, wherein the chamber provides means to control at least one environmental parameter such as temperature or humidity.

The invention further provides bioreactor assemblies (microfermentor arrays) for performing multiple fermentations in parallel. Such assemblies include a plurality of microscale bioreactors as described herein.

In other aspects, the invention includes a variety of methods for using the microscale bioreactors and microscale bioreactor arrays. For example, the invention provides a method of selecting a strain that produces a desired product or degrades an unwanted compound comprising steps of (a) culturing a plurality of different strains, each in an individual microscale bioreactor; (b) measuring the amount of the desired or unwanted product in each of the microscale bioreactors; and (c) selecting a strain that produces an optimum amount of a desired product or degrades a maximum amount of the unwanted compound. The invention further provides a method of selecting a bioprocess parameter comprising steps of (a) culturing an organism type in a plurality of microscale bioreactors, wherein the microscale bioreactors are operated under conditions in which the value of the bioprocess parameter varies and wherein the organism produces a product or degrades a compound; (c) monitoring biomass in each of the microscale bioreactors; and (d) identifying the value of the bioprocess parameter that results in optimum biomass, optimum product formation, or optimum compound degradation. In addition to biomass, other bioprocess parameters may also be monitored, and multiple parameters may be varied. According to certain embodiments of the invention the bioprocess parameter or parameters are actively controlled.

The contents of all papers, books, patents, etc., mentioned in this application are incorporated herein by reference.

### BRIEF DESCRIPTION OF THE DRAWINGS

FIGS. 1A and 1B show top and side views of the design of one embodiment of a microfermentor of the invention.

FIG. 2A shows a side view of an embodiment of a two vessel microfermentor in which the fermentation vessel is in contact with the external environment.

FIG. 2B shows a side view of an embodiment of a two vessel microfermentor in which the fermentation vessel is enclosed.

FIG. 3 (upper portion) shows a design of an embodiment of a microfermentor in which components are provided externally to the microfermentor vessel. FIG. 3 (lower portion) shows a schematic of a microfermentor array of the microfermentors depicted in the upper portion of the figure.

FIG. 4A shows a schematic of a platform for an integrated microfermentor array and associated system components.

FIG. 4B shows a schematic of a platform for a microfermentor array and associated microfluidics in which bioprocess parameters are varied among the individual microfermentors.

FIG. 4C shows a schematic of robotic loading and sampling of a microfermentor array.

FIG. 5 shows a schematic illustration of the formation of an oligo(ethylene oxide) self-assembled monolayer on a metal oxide surface.

FIG. 6 shows a strategy for generating a self-assembled film incorporating a recognition element.

FIG. 7 shows a schematic illustration of a surface-initiated ring-opening metathesis polymerization from a hydrated metal oxide surface.

FIG. 8 shows schematics of straight (top) and serpentine (bottom) waveguides.

FIG. 9 shows an example of a microfabricated heat exchanger.

FIG. 10 is a flowchart of the fabrication procedure employed in one embodiment of the invention.

FIG. 11 shows a top view of a completed microfermentor fabricated as outlined in FIG. 10 and filled with phenol red.

FIG. 12 illustrates a one-dimensional resistance-in-series model of the membrane and the medium, which was used to model oxygen diffusion into a microfermentor.

FIG. 13A shows the calculated steady state oxygen concentration using a one-dimensional resistance-in-series model obtained assuming a cell population homogenously spread throughout the medium.

FIG. 13B shows the calculated steady state oxygen concentration profile using a one-dimensional resistance-in-series model of membrane and medium obtained assuming a membrane thickness of 100 μm, a microfermentor depth of 300 μm, and a cell population of 10¹¹ cells/L, with the cells at the bottom of the microfermentor (heterogenous case).

FIG. 14 shows a schematic of a microscale bioreactor system with associated optical excitation and detection sources.

FIGS. 15A and 15B depicts two views of a microfermentor system in which a microfermentor is placed in an environmental control chamber. The transparent glass slide is not readily visible.

FIG. 16 shows optical density and dissolved oxygen data obtained from batch fermentation of E. coli in a microfermentor in medium without glucose.

FIG. 17 shows optical density and dissolved oxygen data obtained from batch fermentation of E. Coli in a microfermentor in medium containing 30 g/L glucose.

FIGS. 18A and 18B show optical density and dissolved oxygen data obtained from batch fermentation of E. coli in a bench scale fermentor.

FIG. 19 shows a schematic diagram of an embodiment of the invention in which biomass, dissolved oxygen, and pH can be measured simultaneously.

FIG. 20 is a graph comparing pH curves in the microfermentor and in a 0.5 L bench scale fermentor (Sixfors).

FIG. 21 shows a schematic of a microfermentor integrated with optical density, dissolved oxygen, and pH sensors together with associated instrumentation and computer software.

FIG. 22 shows images of cells exposed either to an uncoated glass surface or to glass surfaces that were coated with various comb polymers. The central panel in the upper portion of the figure shows the molecular formula of the polymers.

FIG. 23 shows modeling of oxygen transfer in a microbioreactor as resistances-in-series.

FIG. 24 shows the modeled oxygen concentration profile across PDMS and membrane at t=0, 1, 2 hours (with cell growth modeled as exponential growth).

### DETAILED DESCRIPTION OF CERTAIN EMBODIMENTS

I. Overview

The present invention encompasses the recognition that microscale bioreactors (microfermentors) offer a means of addressing the continuing demand in bioprocess science and engineering for fast and accurate analytical information that can be used to rapidly evaluate the interactions between biological systems and bioprocess operations. In addition, such systems provide a platform for efficiently incorporating modern tools of biology (e.g., genetics, enzymology, molecular biology, and bioinformatics) to improve bioprocess screening and development. For example, microscale bioreactors allow the rapid screening of strains and metabolic pathways for applications ranging from synthesis of natural products to bioremediation. Bioprocess technology has been instrumental in the development and large-scale production of numerous pharmaceuticals and vaccines. In addition, bioprocesses are employed in the food industry, waste treatment, etc.

Metabolic pathway engineering is making a profound impact in areas as diverse as drug discovery (e.g., through the synthesis of novel natural products (2)), commodity chemicals (e.g., the synthesis of ascorbic and lactic acids (3) 1,3-propanediol (4)), and the biodegradation of toxic pollutants (5). Metabolic engineering encompasses the targeted improvement of product formation or cell properties through the modification of biochemical reactions. Hence, metabolic engineering focuses on determining the enzymes that offer the greatest amount of control over the rate of production of a certain metabolite (metabolic control analysis or MCA), then altering the activity of those enzymes (e.g., via molecular biology) and/or altering relevant reaction conditions to manipulate product yields. MCA can involve making mathematical models, carbon tracing, and developing assays for obscure metabolites and aids in the understanding of metabolic fluxes. The alteration of enzyme activities can involve polymerase chain reaction (PCR) techniques, genetic library construction, screening, cloning, and other molecular biology tools. Microfermentor technology will have a significant impact both on how bioprocess development and metabolic engineering research are carried out and also on how rapidly research can be translated into improvements into bioprocesses.

The invention provides microscale bioreactors that include a vessel for culturing cells having a interior volume of less than 200 μl and means for providing oxygen to the interior of the vessel so as to support the growth of cells. The terms “interior volume” and “working volume” are used interchangeably herein. In addition, the invention provides a microscale bioreactor system including a microscale bioreactor and a chamber that provides environmental control. The invention also provides a bioreactor assembly including an array of microscale bioreactors, which may be operated in parallel. The availability of a large number of bioreactors operating in parallel offers a number of unique advantages. For example, the microfermentor array makes it possible to (i) systematically evaluate the effects of varying one or more of a large number of parameters (e.g., temperature, nutrient composition, pH, etc.) on any phenotypic characteristic of interest, e.g., growth rate, metabolite production or compound biotransformation ability, etc., of a particular strain or (ii) systematically evaluate the characteristics (e.g., metabolite production) of a large number of different strains while holding environmental conditions constant.

Developing microscale bioreactors requires more than merely scaling down from currently available fermentor technology. For example, the large volumes employed in traditional fermentors makes it possible to monitor parameters such as oxygen concentration, biomass, etc., by removing samples from the fermentor at appropriate times. Sequential sampling may be impractical in the context of a microscale bioreactor or may need to be performed differently and on a smaller scale. Large indwelling sensor devices are not practical in the context of a microfermentor. Thus accurate monitoring of bioprocess parameters, a requirement for many applications, requires the development of alternative methods. Furthermore, oxygenation using traditional techniques such as sparging and/or stirring may be problematic in small volumes.

In addition to the challenges discussed above, use of fermentors with small volumes offers a number of potential advantages. For example, microfabrication technologies can be used to efficiently produce a large number of identical microfermentors. Microfabrication also allows integration of sensing devices into the structural components of the bioreactor, which enhances the possibilities for acquiring large amounts of data in an efficient manner. Thus in preferred embodiments of the invention at least one sensing device is integrated into a structural component of the microfermentor.

Miniaturization of fermentation processes to microliter scale represents a significant departure from conventional procedures. The inventors have recognized the need to address the following significant issues: (i) design and fabrication techniques, including materials selection and surface modification; (ii) bioprocess parameter control; (iii) selection, development, and integration of sensor technology; and (iv) appropriately sensitive analytical devices. In addition, the inventors have recognized the importance of utilizing appropriate biological systems for evaluating performance of the microfermentors and for comparing microfermentors with traditional bioprocessing methodologies. Significant differences between traditional fermentors and microfermentors include, for example (i) the ratio of wall surface area to volume; (ii) more significant evaporative losses in microfermentors; (iii) incompatibility of microfermentors with conventional oxygenation methods.

As described in more detail in the Examples, the inventors have constructed a microscale bioreactor with a working volume of 5 μl and have shown that it can support the growth of bacterial cells. At the end of the fermentation run, which lasted greater than 10 hours, the cells were still viable. Results indicate that cell growth in the microfermentor is comparable to cell growth in a conventional fermentor. The inventors have demonstrated successful delivery of oxygen to the microfermentor interior and lack of toxicity.

The following sections provide relevant definitions, describe the manner in which the invention addresses the foregoing concerns and others, and describe methods for using the microfermentor and microfermentor arrays of the invention.

II. Definitions

Bioreactor Operation Strategies: In accordance with the terminology as commonly accepted in the art and described in (54), bioreactor operation strategies can be classified into one of three general modes, i.e., batch or fed-batch operations, the semi-continuous or cut-and-feed strategy (which may also be referred to as semi-batch), and perfusion culture. Batch culture is usually performed using suspension culture cells in a stirred tank bioreactor, although in the case of a microreactor as described herein, stirring may or may not be performed. Product is harvested from the medium at the end of the batch cycle. Fed-batch culture differs from batch culture in that nutrients are added either continuously or periodically during the batch cycle. The semi-continuous or cut-and-feed strategy also typically employs stirred tank, homogeneously mixed bioreactors. In this operating strategy a bioreactor is inoculated with cells, which are then allowed to grow for a period of time, often until the culture is approaching early stationary phase. A large fraction of the cell culture broth is then harvested, usually on the order of 70-90%, and the bioreactor replenished with fresh medium. The cycle is then repeated. Perfusion operations retain cells within the reactor while allowing a cell-free sidestream to be removed; they can be subdivided into two categories, the homogeneous systems such as the perfusion chemostat or heterogeneous systems like hollow fiber or fluidized bed bioreactors. It is to be understood that these definitions are not intended to limit the invention or its modes of operation in any way and that they are to be interpreted as appropriate in the context of microfermentors as described herein.

Channel: The term “channel” refers to a hole of constant or systematically varied cross-sectional area through a material. Generally a channel has a defined cross-sectional geometry, which may be rectangular, ovoid, circular, or one of these geometries with an imposed finer feature, such as indentations, etc.

Fermentation: The terms “ferment”, “fermentation”, etc., are to be understood broadly as indicating culture of cells in general. The terms do not imply any particular environmental conditions or metabolic processes. While typically these terms refer to culture of bacterial cells (e.g., eubacteria), they may also apply to archaebacteria or eukaryotic cells (e.g., yeast or mammalian cells). As a noun, a “fermentation” or “fermentation run” or “fermentor run” refers to a period of time during which cells are cultured in a fermentor.

Microscale bioreactor: As used herein the term “microscale bioreactor” is used to describe a bioreactor (i.e., an apparatus for culturing cells) having an interior volume of less than 1 ml. The terms “microscale bioreactor” and “microfermentor” are used interchangeably herein.

Parallel: Fermentor runs are performed “in parallel” when the run times of the fermentor runs overlap. The runs may, but need not be, started and/or terminated at substantially the same time. The runs may last for the same length of time or for different lengths of time.

Strain: In a broad sense, cells or viruses may be considered to be of different strains if they differ from each other in one or more phenotypic or genotypic characteristic. In general, a “strain” is a population of organisms descended from a single cell and maintaining the phenotypic and genotypic characteristics of that cell. Although frequently used to refer to microbes (i.e., microscopic organisms), the term may be used herein to refer to cells of any type.

III. Design and Fabrication

A. Design

In certain embodiments of the invention the microscale bioreactor comprises a vessel for culturing cells and a means for providing oxygen to the vessel at a concentration sufficient to support cell growth. In certain embodiments of the invention the vessel has an interior volume of less than 1 ml. In certain embodiments of the invention the vessel has an interior volume of less than 200 μl. In certain preferred embodiments of the invention the working volume is between 50 μl and 100 μl inclusive. In certain preferred embodiments of the invention the working volume is between 5 μl and 50 μl, inclusive. In certain preferred embodiments of the invention the working volume is between 5 μl and 10 μl, inclusive. In certain preferred embodiments of the invention the working volume is approximately 7.5 μl or approximately 10 μl. In certain preferred embodiments of the invention the working volume is approximately 5 μl. (Generally the term “approximately” as used herein will indicate that a number may vary by ±1%, ±5%, ±10%, depending upon the context.) Small working volumes offer a number of advantages. For example, they permit efficient gas-liquid contacting to control the level of dissolved oxygen (DO). Small working volumes also imply smaller diffusion times, which aids in exchange of gases. In addition, microscale bioreactors having working volumes in the range of between 5 μl and 50 μl or between 50 μl and 100 μl may be more easily produced using microfabrication than those with larger working volumes. Microfabrication facilitates the production of microfermentor arrays with a very high density of individual microfermentors. In addition, microfabrication allows for configurations with very large specific gas-liquid interfaces. Particularly in the context of microscale bioreactors employing active aeration, microfabrication allows one to achieve a large mass trans coefficient (k_{L}a). For example, the inventors have achieved a greater than two orders of magnitude increase in mass transfer coefficients for gas-liquid-solid reaction systems by precise design of the contacting scheme (8). Moreover, small system dimensions imply faster diffusion across the vessel volume and thus more uniform conditions within. Furthermore, smaller dimensions (e.g., dimensions resulting in an interior volume of less than approximately 100 μl) may be desirable to ensure adequate support for an aeration membrane that forms the top of the culture vessel.

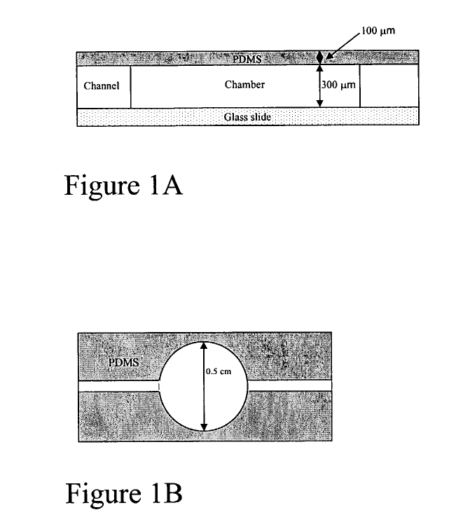

FIGS. 1A and 1B show top and side views of the design of one embodiment of a microfermentor of the invention. As seen in FIG. 1A, in this embodiment of the invention the vessel has a round cross-section in the horizontal dimension with an overall cylindrical configuration. The bottom of the microfermentor is formed from a rigid substrate (e.g., silicon, glass, plastics such as polycarbonate, plexiglass, etc.), sufficiently strong to support and stabilize the remaining portions of the structure. In certain embodiments of the invention at least one wall (e.g., a side wall, top wall, or bottom wall) of the microfermentor comprises a transparent material to permit optical access. However, in certain embodiments of the invention use of a transparent material is not necessary as waveguides can be used to guide light in or out (see below).

As shown in FIG. 1, in preferred embodiments of the invention one or more channels extend from the vessel. For example, in those embodiments of the invention that operate in batch mode, the channels are used solely to introduce medium and inoculum (i.e., cells) to the vessel prior to the beginning of a fermentation. However, in certain embodiments of the invention such channels may be used for other purposes, e.g., to remove samples, to introduce additional components such as nutrients, buffers, etc., during the course of a fermentation. The channels may conveniently be used to interface with robotics, e.g., for introducing components into the vessel and/or for removing samples. Robotics may be used, for example, to interface microfermentors or microfermentor arrays with, for example, a microtiter plate from which materials may be transferred into the fermentor or into which samples may be placed. The channels may connect with pumps, reservoirs, etc. Microfluidics technology may be employed.

As described further below, the microfermentor includes means for delivering oxygen to the vessel. In preferred embodiments of the invention one or more walls of the microfermentor vessel consists at least in part of a gas-permeable membrane for oxygenation of the growing culture. The gas-permeable membrane may also aid in dispersal of gases produced during metabolism. In certain embodiments of the invention as described in Example 1, the membrane serves as both the aeration membrane and the structural material of the microfermentor. For example, as shown in FIG. 1, both the top and side walls of one embodiment of the microfermentor are made of the polymeric material poly(dimethylsiloxane) (PDMS). In certain embodiments of the invention the microfermentor includes multiple membranes. These membranes may be made from the same material or from different materials, e.g., materials having different properties such as gas diffusivity and solubility.

Since adequate oxygenation is a major consideration for cell growth, selection of appropriate microfermentor dimensions and membrane materials may be guided by an oxygen transport model that takes into account the properties of the oxygen delivery system. Use of such a model is described in more detail in Example 2. The calculations therein may readily be applied to any given material for which parameters such as oxygen diffusivity and solubility are known. In certain embodiments of the invention the permeability (i.e., product of diffusivity and solubility) of the membrane to oxygen is approximately equal to that of PDMS, i.e., 800 Barrer (1 Barrer=10⁻¹¹ cm³(STP)·cm/cm²·s·cm Hg) (44). In certain other embodiments of the invention the permeability of the membrane to oxygen is greater than 800 Barrer. In certain other embodiments of the invention the permeability of the membrane to oxygen is either between approximately 600 and 800 Barrer, between approximately 400 and 600 Barrer, between approximately 200 and 400 Barrer, or between approximately 80 and 200 Barrer.

The invention provides a variety of microscale bioreactor systems in which two vessels are separated by a membrane. A first vessel serves as a cell culture vessel while the second vessel contains a liquid that serves as a source of one or more components such as oxygen, nutrients, buffers, etc. A variety of different configurations are possible.

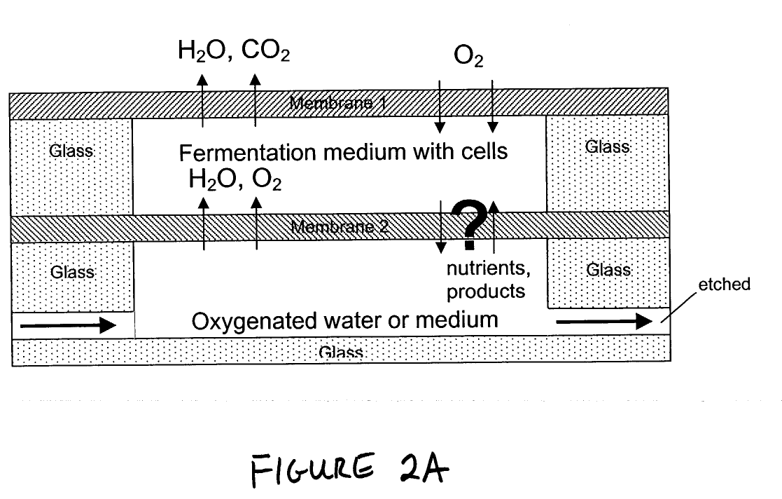

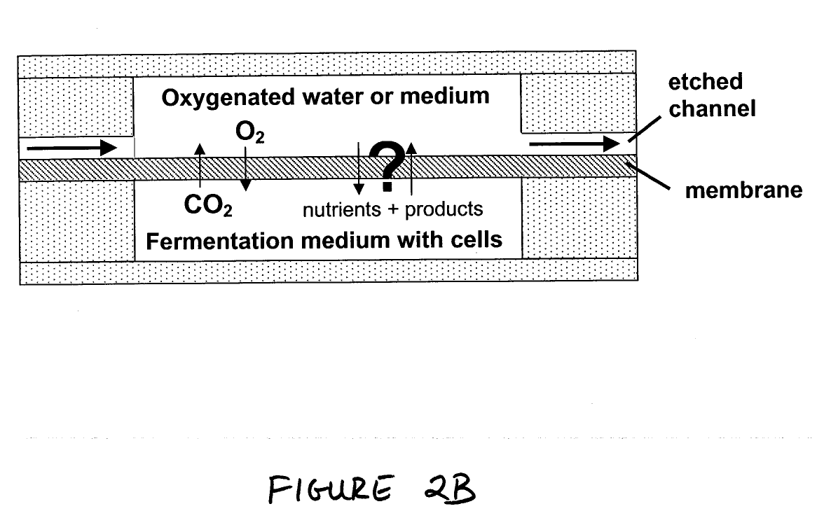

FIG. 2A shows a side view of one such embodiment of the invention in which the fermentation vessel is on top. The two vessels of the microscale bioreactor are separated by a membrane (Membrane 2) that allows free transport of water and oxygen into the top vessel. In certain embodiments of the invention this membrane prevents back-diffusion of nutrients, products, and/or salts while in other embodiments of the invention the membrane is permeable to these components. (The question mark in the figure indicates that nutrients, products, and salts may or may not diffuse through Membrane 2.) Membranes such as those typically used in desalination applications can be used for this purpose. A wide variety of membranes that may be used to control the transport of nutrients, products, salts, and cells is available from, e.g., Millipore Corp., Bedford, Mass. Factors such as pore size, surface characteristics such as hydrophobicity, and presence of channels for active or passive transport may be selected by one of ordinary skill in the art to achieve desired transport characteristics.

In the design depicted in FIG. 2A the top membrane (Membrane 1) allows diffusion of water and gases. Salts are not volatile so will not evaporate from the top membrane (Membrane 1), while most products are too large to diffuse readily through the top membrane. Channels in communication with the lower vessel allow oxygenated water to flow through the lower vessel, providing a continuous supply of oxygen and water to diffuse across Membrane 2. Circulation may be achieved using a pump. Since the liquid circulates and can be replenished, the volume of the lower vessel may be small relative to the volume of the upper vessel and may, in certain embodiments of the invention, consist merely of a chamber with similar height to that of the channels.

In certain embodiments of the invention rather than circulating liquid through a lower vessel as shown in FIG. 2A, a lower vessel with a volume that is large relative to the volume of the upper vessel (e.g., at least twice the volume of the upper vessel) is used, thus providing a reservoir of component(s). The contents of the reservoir may be replaced periodically. There may also be channels (not shown) in communication with the cell culture vessel, e.g., in order to allow introduction of cells and culture medium, removal of samples, etc.

This design offers the following features and advantages, among others: (1) Water losses from evaporation may be replaced by osmosis from bottom vessel; (2) Oxygenation may be provided from both the top and bottom (increases maximum allowable depth); (3) Contact with large reservoir of pH-neutral water or medium allows neutral pH to be maintained in the fermentor; (4) The process remains batch if only gases and water permeate membrane, while if the membrane allows nutrients, products, etc., to also permeate, process becomes semi-batch or continuous; (5) Since sensors may be integrated onto the glass or other material from which the microfermentor is fabricated, they are now separated from the fermentation medium. This allows separate calibration for sensors, and also eliminates need to sterilize sensors (e.g. some sensors are UV or temperature sensitive); (6) The design allows control of the oxygen gradient within the culture vessel by controlling oxygen content of water below, and atmosphere above, the culture vessel.

FIG. 2B shows another embodiment of a two-vessel microfermentor design. In this embodiment the culture vessel is not in contact with air. Instead, oxygen is provided via a membrane that separates the culture vessel from a second vessel that contains a reservoir of oxygenated liquid, e.g., water. The separating membrane allows free transport of water and oxygen into the culture vessel. In certain embodiments of the invention this membrane prevents back-diffusion of nutrients, products, and/or salts while in other embodiments of the invention the membrane is permeable to these components. (The question mark in the figure indicates that nutrients, products, and salts may or may not diffuse through the membrane.) Oxygenated liquid may be flowed through the upper vessel via channels as shown. In this design diffusion from the upper to the lower vessel takes place in the same direction as the gravitational forces.

This design offers the following features and advantages, among others: (1) Water losses from evaporation may be eliminated by contact with the water-filled vessel; (2) Contact with a large reservoir of pH-neutral water or medium allows neutral pH to be maintained in the fermentor; (3) The process remains batch if only gases and water permeate membrane, if the membrane allows nutrients, products, etc. to also permeate, process becomes semi-batch or continuous.

Although in FIGS. 2A and 2B the permeable membranes separating the two vessels have been depicted as structural components of the vessels, this need not be the case. The permeable membranes may instead form a portion of a separating layer made from a less permeable material.

In summary, the two-vessel designs address the potential problem of evaporative losses that may occur, e.g., in a non-humidified environment. In addition, these designs provide a second source of oxygen for the fermentation, and as a result a deeper culture vessel with a larger volume to surface ratio can be utilized. These designs also allow for control of pH, e.g., by allowing diffusion of protons and hydroxyl ions. In addition, pH control may be enhanced by providing appropriate buffers in the liquid that fills the second (non-culture) vessel.

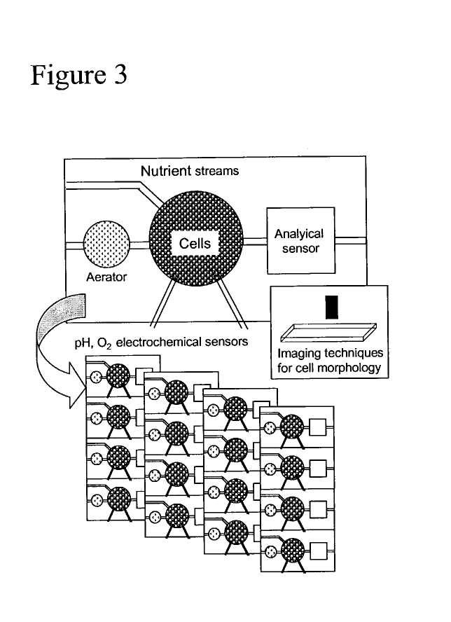

FIG. 3 shows a design of yet another embodiment of a microfermentor. The upper portion of FIG. 3 shows a single microfermentor unit. Each microfermentor includes a vessel in which cells are cultured and multiple channels extending from the vessel. The channels allow nutrient streams to enter the vessel and also provide means of contact between the interior of the vessel and various sensor devices. In this embodiment of the microfermentor, aeration is provided by means of a channel that allows communication between the microfermentor vessel interior and an external aeration chamber. This chamber may, for example, connect to a source of oxygen, may include a stirrer, etc. Multiple individual microfermentor units may be connected to a single aerator or each unit may have a dedicated aerator unit.

One of the goals of the invention is to provide an efficient platform in which multiple fermentations can be performed in parallel (e.g., simultaneously). Accordingly, the invention provides a system comprising a microfermentor array, by which is meant a plurality of physically connected microfermentors. The microfermentors are typically arranged in a regular geometry such as in mutually perpendicular rows, but this is not a requirement. Microfermentors are understood to be “physically connected” if they are arranged on or in a single substrate, attached to a common base, and/or connected to each other or to a central receptacle or chamber (e.g., via channels). The microfermentor arrays may include any number of individual microfermentor units. For example, in certain embodiments of the invention a microfermentor array includes at least 10 microfermentors. In certain embodiments of the invention a microfermentor array includes at least 100 microfermentors, at least 1000 microfermentors, or at least 10,000 microfermentors. The lower portion of FIG. 3 presents a sketch of an embodiment of a microfermentor array in which the individual microfermentor units shown in the upper portion of FIG. 3 are employed. (For illustrative purposes the columns are offset from one another.)

According to certain embodiments of the invention the system consists of multiple microfermentors, each with integrated bioanalytical devices, and operating in parallel. This system addresses the continuing demand in bioprocess science and engineering for fast and accurate analytical information that can be used to rapidly evaluate the interactions between biological systems and bioprocess operations. Moreover, the microfermentors provide the platforms for efficiently incorporating modern tools of biology (e.g., genetic profiling, enzyme catalysis, and bioinformatics) to improve bioprocess screening and development.

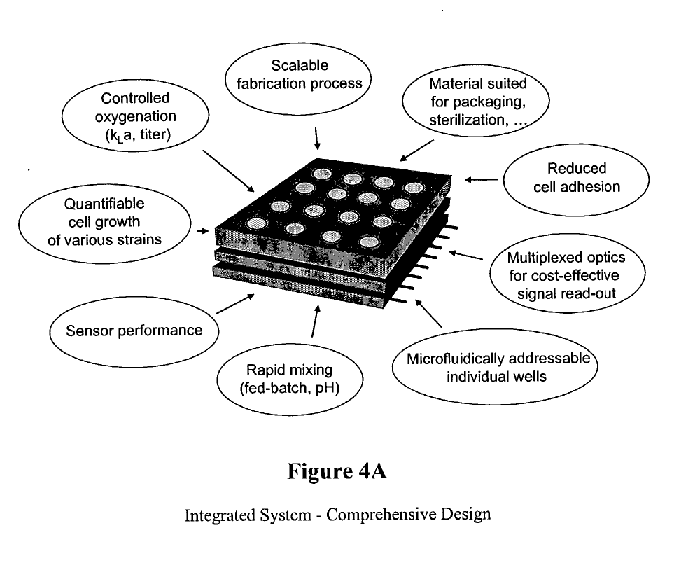

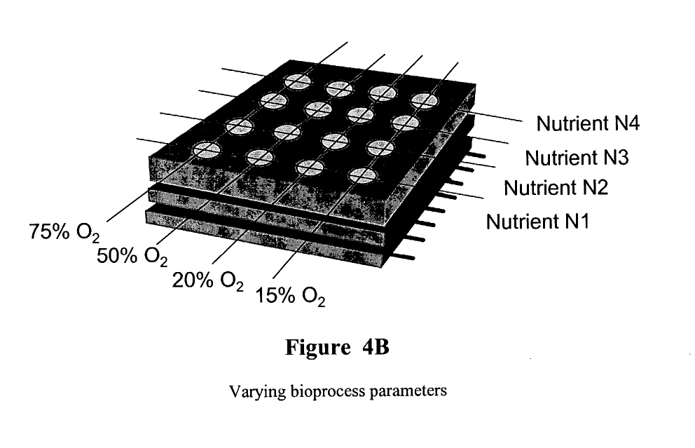

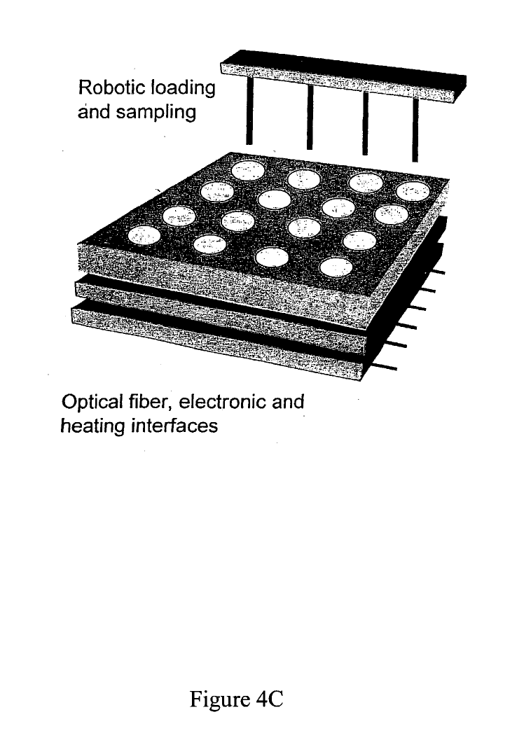

FIG. 4A is a schematic diagram of a system comprising an array of microfermentors consisting of mutually perpendicular rows and columns of individual units. Any of the microfermentors described herein may be either placed within the wells of the plate depicted in FIG. 4A or the wells themselves may serve as individual microfermentor vessels. According to certain embodiments of the invention the system allows for integrating parallel operation of multiple microfermentors with fluid delivery and optical and electronic sensing elements. The microfermentors can be run in different modes including batch, fed batch, and continuous. According to certain embodiments of the invention the microfermentor units can be autoclaved and exchanged.

The plate has chambers for multiple, parallel fermentation experiments. As shown in FIG. 4B, fluidic interface elements needed, for example, to inoculate the culture medium, to control pH, to add nutrient(s), or to remove portions of the cell culture may be integrated on the plate and in the system interface. This integration may be performed in such a way as to minimize mechanical manipulations and components needing sterilization. Elements present on or in the plate would typically include simple channels, valves, and connections to the system interface, etc. Other elements may also be included. Fluid control elements and delivery methods (e.g., pumps) may be housed in the system itself.

Similarly, according to certain embodiments of the invention reusable sensing elements are located elsewhere within the system whereas one-time use components are incorporated on or in the plate. For example, fluorescent dyes for dissolved oxygen and pH measurements may be incorporated into the plate, whereas optical fibers, lenses, and optical detection equipment may be situated in the system interface so that they could be used repeatedly for successive fermentation experiments. According to certain embodiments of the invention other means, e.g., optical means for measuring fluorescence and luminescence from biological species are incorporated into the system as described herein. Analogously, according to certain embodiments of the invention electronic sensing and automation means are incorporated into the system itself whereas simple actuator and sensing elements (e.g. electrochemical and capacitance) are incorporated into the plate.

According to certain embodiments of the invention the plate is packaged at the point of manufacture and may be pre-sterilized. When starting parallel fermentation, the plate is removed from the package and easily mounted in the system.

The plate and/or other system components can be manufactured by any of a number of standard microfabrication techniques, or combinations thereof, including but not limited to hot embossing, injection molding, electroplating, microelectrode discharge machining etc. According to various embodiments of the invention the plate is disposable or reusable depending, for example, on the particular application.

FIG. 4B is a schematic diagram of a system comprising a microfermentor array with microfluidic channels allowing control over parameters in individual microfermentors (see discussion of bioprocess control below). According to the approach depicted in FIG. 4B, by varying each of multiple parameters across different dimensions of the array, a combinatorial effect is achieved. For example, by employing four different values for dissolved oxygen and four different nutrient compositions across the two dimensions of the array, a total of 16 different culture conditions may be tested. According to various embodiments of the invention a single bioprocess parameter is varied across a single dimension of the array. According to certain other embodiments of the invention a plurality of bioprocess parameters are varied across one or more dimensions of the array.

Microfermentor arrays in which a plurality of substantially identical microfermentors operate in parallel offer a number of advantages. For example, it is possible to operate multiple microfermentors in parallel, terminate the fermentor run of one or more microfermentors at each time point of interest, and subject much or all of the contents of the microfermentor(s) to analysis. This offers an alternative to the approach of removing multiple samples from a single microfermentor, as would typically be done with a traditional bench-scale or industrial scale fermentor (although this approach may also be employed in the case of a microfermentor of the invention). The availability of multiple microfermentors operating in parallel thus offers higher flexibility for analysis.

The possibility of operating multiple microfermentors in parallel means that it will be possible to conveniently perform multiple substantially identical fermentation runs (e.g., multiple runs under identical or substantially identical conditions and/or in which the same organism is used) and to analyze the results of multiple such fermentation runs, which can greatly enhance confidence in the results. The degree to which conditions must be similar in order to be considered “substantially identical” may vary depending on the application and the particular condition under consideration. For example, two fermentation runs may be considered to occur under “substantially identical conditions” with respect to a particular parameter if the parameter varies between the two runs by less than approximately 20%, less than approximately 10%, less than approximately 5%, less than approximately 1%, or less than approximately 0.1%, depending, e.g., upon the particular parameter, the purpose of the fermentation run, etc. Rather than relying on results obtained from one or even a few large fermentations, the microfermentor arrays of the invention offer the possibility of obtaining data with increased statistical significance and of reliably identifying trends and variations, e.g., caused by different culture conditions.

In certain embodiments of the invention the microfermentor(s) and/or sensor(s) interface with standard laboratory robotics, with analytical equipment (e.g., HPLC, GC/MS, FTIR, etc.) and/or with data acquisition systems. In particular, in certain embodiments of the invention interfacing optical microscopy with the cell unit allows optical monitoring of cell morphology. In certain embodiments of the invention the microfermentors and microfermentor arrays are disposable.

The microfermentors, microfermentor arrays, and microfermentor systems of the invention may be mounted on or attached to a base and/or enclosed within appropriate housing. The housing may be provided with access ports, e.g., to allow entry and exit of wires, cables, tubes, etc. As used herein, according to various embodiments of the invention a “microfermentor system” includes one or more microfermentors or microfermentor arrays as described herein, optionally with associated microfluidic components, and one or more of the following: a plate or platform on or in which one more microfermentors or microfermentor arrays, optionally with associated microfluidics, may be mounted or housed; a chamber in which the microfermentors or microfermentor arrays, plates, or platforms may be enclosed; a pump; sensing and/or detection means; analytical equipment; robotics; software and computers, e.g., for data acquisition and/or bioprocess control; and any wires, cables, fibers, electronic components, etc., needed for operation of any of the foregoing system components. The system may include means for delivering energy to any component of the system, e.g., a power supply, and/or means for delivering excitation such as light or other forms of electromagnetic energy to the system.

B. Fabrication Techniques

A wide variety of fabrication techniques may be used to construct the microfermentors of the invention. As described in more detail in Example 1, in certain embodiments of the invention microfabrication using soft lithography is employed. This technique offers a number of advantages. For example, soft lithography allows the rapid production of microfermentors with different shapes and sizes, allowing efficient optimization of these parameters.

In certain embodiments of the invention, e.g., for purposes of large scale manufacture it may be preferable to select alternative techniques or materials. For example, in certain embodiments of the invention the microfermentor is fabricated at least in part from a polymeric material such as polystyrene, polycarbonate, polypropylene, or polytetrafluoroethylene (TEFLON™), copolymers of aromatics and polyolefins, which can be processed using standard methods such as free-form molding, micromolding, injection molding (e.g., reaction or thermoplastic injection molding, punching, etc.), hot embossing, CNC machining, laser direct write, microelectrodischarge machining, etc. See, e.g., (78). An aeration membrane can be incorporated as a structural component of the microfermentor vessel or into a vessel wall. Incorporation may occur during fabrication of the remainder of the vessel or the aeration membrane may be added later. For example, an aeration membrane may be attached using any of a variety of techniques, e.g., with adhesive, heat fusion, etc.

In certain embodiments of the invention the microfermentors and microfermentor arrays are fabricated using standard semiconductor manufacturing technology as described, for example, in (77). For example, a silicon wafer (which may be mounted on a rigid substrate such as glass or plastic) may be used to form the lower layer of the microfermentor, which can then be etched to form a well that functions as a vessel for growth of cells. Additional layer(s) of semiconductor materials such as silicon nitride may be deposited on the lower layers (e.g., by chemical vapor deposition, physical vapor deposition, and electrodeposition), with wells and channels etched into one or more of these layers. As described above, a microfermentor array including multiple wells can be formed, and the wells may be connected via channels to each other, to the edge of the wafer, or to a central receptacle, which may be used to supply nutrients, oxygen, or cells to the interior of the well and/or to remove samples.

In certain embodiments of the invention a manufacturing technique that allows substantially integrated and simultaneous fabrication of some or all of the structural components of the microfermentor (i.e., components such as bottom, top, and side walls necessary to form a vessel within which cells can be cultured) and one or more functional components (e.g., oxygen delivery means, sensors, etc.) is selected. In certain embodiments of the invention a manufacturing technique is selected that allows fabrication of some or all of the structural components of the microfermentor directly on a substrate or base. Such an approach contrasts, for example, with a manufacturing technique in which it is necessary to fabricate part of the vessel (e.g., the side walls) and then attach it to a base.

C. Materials and Surface Modification

In certain preferred embodiments of the invention biocompatible materials (i.e., materials that will not significantly inhibit or adversely affect cell viability and proliferation and/or adversely affect other biological components such as metabolites produced by the cells) are employed for those portions of the microfermentor that are in contact with cells or are used to deliver cells or other materials to the vessel. Suitable materials include silicon, silicon dioxide (e.g., glass), ceramics, plastics such as polycarbonates, acrylates, polypropylenes, polyethylenes, polyolefins, or other biocompatible polymers such as silicones (for example, PDMS), fluoropolymers, etc. In addition, nonbiocompatible materials (e.g., certain metals) can be employed provided they are coated with a biocompatible material.

PDMS represents an attractive choice for microfermentor fabrication (both for the aeration membrane and as the structural material of the microfermentor itself) for a number of reasons. PDMS is highly permeable to gas, which allows sufficient oxygen to diffuse into the medium while simultaneously allowing carbon dioxide and other gases to escape. PDMS is highly hydrophobic, which minimizes water loss to evaporation. It is biocompatible, can withstand autoclaving temperatures, and is transparent to visible light.

The small sizes of the microfermentors and the other features within these systems lead to surface-to-volume ratios that are well above those in conventional macroscale operations, accentuating the importance of providing compatible interfaces for operation. Protein denaturation and non-specific adsorption provide pathways that could potentially alter the performance of the microfermentors. Thus in certain embodiments of the invention surfaces in contact with cells and/or biological components such as metabolites produced by the cells are altered in order to reduce these effects. Such surfaces may include both the interior of the microfermentor vessel and any channels, etc., that may contact either cells or other biological components such as cell products.

In certain embodiments of the invention surfaces in contact with cells or other biological components are altered in order to inhibit or promote cell adhesion. For example, in the case of bacterial cells, cellular adhesion to microfermentor surfaces is undesirable and surfaces in contact with cells may therefore be modified to reduce cell adhesion. Similarly, adhesion of cell products such as proteins may be undesirable. Adhesion may reduce the efficacy of aeration membranes and the accuracy of sensors. In addition, adhesion may contribute to denaturation of cell products and difficulty with efficient collection of such products.

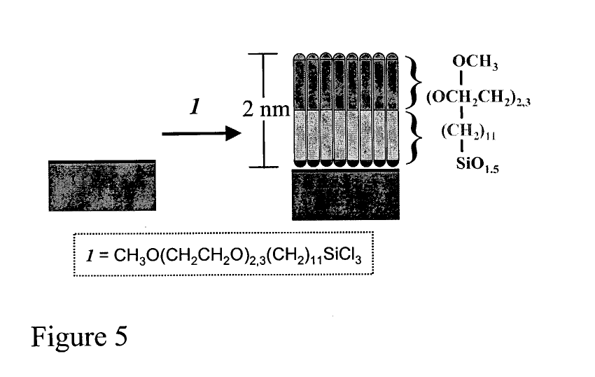

To alter the adsorptive properties of the contacting surfaces of the microfermentor and any connecting microchannelled networks toward the various biological components of the system a number of different approaches may be employed. In certain embodiments of the invention the surfaces are coated with a polymer. In certain embodiments of the invention the surfaces are derivatized with self-assembling molecular films prepared from CH₃O(CH₂CH₂O)ₙ(CH₂)₁₁SiCl₃ (n=2-4) (as described in 14). These reagents produce an oriented chemisorbed monomolecular film on the surfaces of metal oxides. These films are densely packed and expose oligo(ethylene oxide) units at the surface that provide a moderately hydrophilic interface with a low interfacial energy with water. See FIG. 5. A notable feature of these films is that they are able to retard the non-specific adsorption of proteins (such as insulin, albumin, lysozyme and others) and oligonucleotides, and to greatly diminish the adsorption of cells.

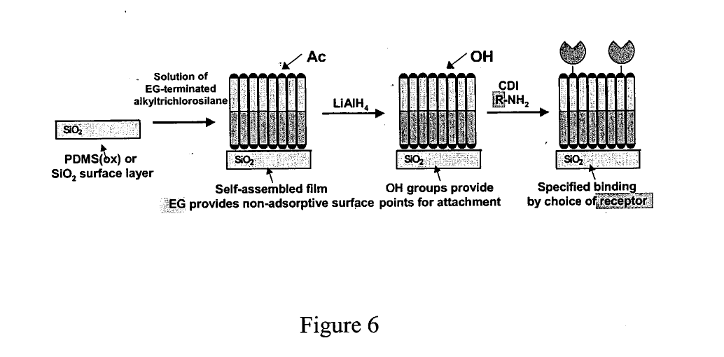

Further reductions in the adsorptive properties of cells may be achieved by the generation of more hydrophilic surfaces (i.e., surfaces with an even lower interfacial energy with water) and a greater entropic contribution against adsorption. Strategies for the production of such surfaces include the use of an acetate-terminated oligo(ethylene oxide) silanating reagent that is then deprotected on the surface to reveal hydroxyl groups or the use of reagents with longer oligo(ethylene oxide) chains. For example, the reagent CH₃CO₂(CH₂H₂O)₃(CH₂)₁₁ SiCl₃ assembles to form an acetate-protected oligo(ethylene glycol) surface which, upon deprotection with LiAlH₄ produces a glycol termination. This surface presents a lower interfacial energy with water, decreases unwanted non-specific adsorption events, and offers a reactive alcohol terminus that inventors have employed to immobilize a protein through coupling using carbonyl diimidazole. See FIG. 6.

A complementary strategy for derivatizing the surfaces is the reaction between Grignard reagents (RMgBr) and a hydrogen-terminated silicon surface (15, 16). The latter is readily formed by treating a silicon surface with hydrofluoric acid. This reaction produces grafted organic chains that are connected to the surface by robust silicon-carbon bonds. This strategy offers a compatibility with basic solutions and a broader set of processing steps than do the use of silanating reagents. According to certain embodiments of the invention in which such films are employed, some amount of surface functionalization is performed during the fabrication process (particularly prior to wafer bonding steps), thereby providing possibilities for generating patterned surfaces within chips. Further, this reaction works well with porous silicon supports and offers the possibility for modifying high surface area regions within a system (9), offering a means to tailor the properties of gas-liquid interfaces used for aeration.

According to certain embodiments of the invention a surface-initiated polymerization process using ring-opening metathesis polymerization (ROMP) is used as a means to produce thicker grafted films onto surfaces (17) and to incorporate functional groups into the films. These films form at room temperature and have thicknesses that can range from 10 to 100 nm, depending on the reaction time. Briefly, the inventors used norbornenetrichlorosilane (NTCS) to assemble a monolayer coating on an oxide surface. Exposure of this primer layer sequentially to a catalyst solution and then a monomer solution resulted in formation of adherent polymer films with thicknesses of tens of nanometers. By employing NTCS as monomer in this polymerization reaction, polymeric films containing reactive functional groups were generated. The side chain trichlorosilane groups have been reacted with poly(ethylene glycol)s (PEG) to generate grafted chains of this polymer on various oxide supports. For example, in one embodiment of the invention films were treated with a 300 molecular weight PEG and then with ethylene glycol. Variants and derivatives of PEG may also be used. According to certain embodiments of the invention methoxy-capped PEGs are used.

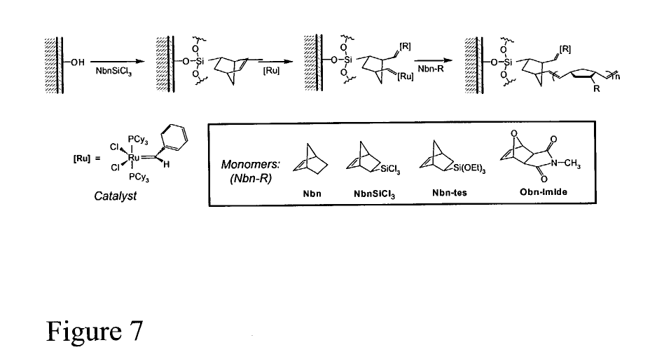

The fact that ROMP chemistry allows a wide range of functionalities to be introduced into the films offers a synthetic flexibility and ease for accessing a broader range of surfaces, and an ability to introduce various amino acids or sugars as components within the coatings. In certain embodiments of the invention this chemistry is used to fabricate more robust coatings on the microfermentor and/or channel inner surfaces and to introduce and control a range of interfacial properties. FIG. 7 shows a schematic illustration of a surface initiated ROMP from a hydrated metal oxide surface. The surface is first derivatized to expose norbornenyl groups then treated to immobilize the [Ru] catalyst. When this surface is treated with a monomer solution, a ROMP polymer grows as a grafted fihn from the substrate.

According to another approach, polymers such as comb polymers (i.e., polymers that comprise polymer side chains attached to a polymer backbone) are allowed to adsorb to the surface or otherwise applied to the surface. In certain preferred embodiments of the invention the backbone of the comb polymer is selected to adsorb to the surface to be coated, and the side chains are selected to retard the adsorption of proteins and/or cells. Appropriate selection of the backbone polymer will, in general, thus depend on the particular surface to be coated. For example, in certain embodiments of the invention in which the surface is glass, variants of a polymer that includes poly(acrylic acid) as a backbone are prepared and grafted with chains of either homogenous PEG or a polymer such as poly(ethylene glycol-r-propylene glycol), containing a heterogenous mixture of molecules. The side chains may thus be identical or nonidentical.

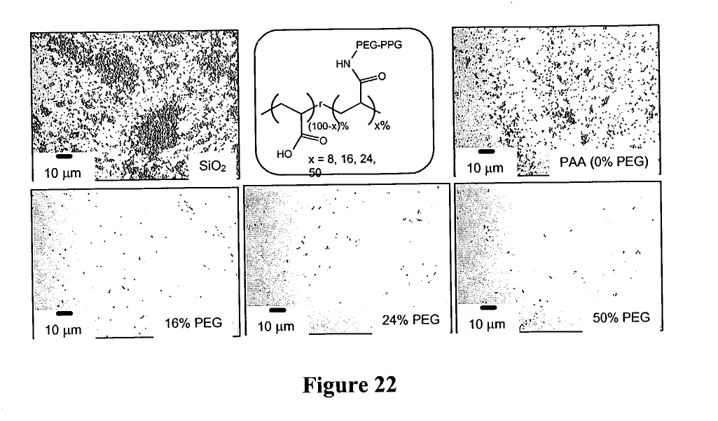

FIG. 22 shows the striking differences in cell behavior when E. coli were exposed to a bare glass surface (upper left panel) as compared with cell behavior when exposed to glass surfaces that had been treated with comb polymers having a poly(acrylic acid) backbone and a range of different PEG contents as indicated (0%, 16%, 24%, 50%). Cells were cultured in bench-scale bioreactors for 3 days in the presence of uncoated glass surfaces and glass surfaces that were coated with the various comb polymers. As is evident from FIG. 22, the presence of the comb polymers greatly decreased cell adsorption. The molecular formula of the comb polymers is presented in the upper center of the figure. The percentage number corresponds to the percent of CO₂H groups (on average) on the poly(acrylic) acid backbone that contained the PEG-PPG graft. For example, if the poly(acrylic acid) molecule comprised 100 monomer units of acrylic acid in its structure, 16% indicates that each polymer molecule contains (on average) 16 CO₂H groups with amide links to a PEG-PPG polymer chain and 84 free underivatized CO₂H groups.

The inventors have recognized that an advantage of using these various chemical processes for tailoring the coatings on the inner surfaces is that they can be formed on the fabricated systems by simply flowing a solution of the required species through or over the device. Control over the fluidics can allow different devices (or portions of a device) to express different surface chemistries. For example, it may be desired to produce distinct regions that have a low interfacial energy with air (such as for aeration operations), that have a low interfacial energy with water (where protein and cellular adsorption is to be minimized), and that provide immobilized recognition elements for the directed adsorption of certain species (such as for sensing operations).

Self-assembly provides a powerful strategy for controlling and monitoring operations within microfabricated devices. Differences in surface reactivity (for metals vs. oxides vs. for silicon) and the abilities to direct the fluidic movements of reactants to specific regions of a device provide the ability to generate the complex patterns and progressions of surface chemistry within these microscale bioreactors for achieving the desired biochemical operation.

In contrast to bacterial cells, in the case of certain mammalian cells adhesion to a substrate promotes cell growth and may even be essential. Thus in those embodiments of the invention optimized for growth of mammalian cells, surface modifications to promote cell adhesion may be employed. In certain embodiments of the invention some surfaces or portions of surfaces are modified so as to reduce adhesion of cells, proteins, etc., while other portions are modified so as to increase adhesion. U.S. Pat. No. 6,197,575 describes various surface modifications that may be used to promote or inhibit the attachment of cells, proteins, etc., and also contains descriptions of various manufacturing techniques.

A variety of other approaches to modification of surfaces may be employed. For example, two or three dimensional stamping or contact printing may be used instead of or in conjunction with the methods described above. (See, e.g., U.S. Pat. No. 5,512,131, WO 96/29629, U.S. Pat. Nos. 6,180,239, 5,776,748). Alternatively, chemical vapor deposition, may be employed. Chemical vapor deposition allows the formation of films in the gas phase and is applicable to three dimensional devices. Among other advantages, it permits deposition of films in cavities. See, e.g., (79) and U.S. Ser. No. 09/912,166 describing chemical vapor deposition of various polymer materials (e.g., paracyclophanes) onto a variety of substrates including polyethylene, silicon, gold, stainless steel, and glass. The polymer may be a reactive polymer and/or a functionalized polymer. In certain embodiments of the invention a surface of the microfermentor vessel and/or channel(s) is coated with a polymeric material, which may incorporate a ligand. The ligand may promote or inhibit the adhesion of cells or molecules.

IV. Sensor Technology

Research in the field of bioprocess monitoring frequently aims at the rapid acquisition of accurate analytical information that can be utilized to optimize cultivation conditions, cultivation times, and product harvesting times, in order to reduce the cost and time required to establish the process. In addition, as most modern industrial bioprocesses are microbial batch or continuous-fed batch cultivations, where control of parameters is required to maintain an optimized process, on-line monitoring of the process is highly desirable. In order to optimize bioprocesses and to perform optimized bioprocesses it is desirable to be able to monitor a variety of parameters including, but not limited to, biomass and environmental variables (e.g., pH, oxygen concentration, metabolite concentration) during the course of a fermentation, for example to allow selection of fermentation conditions that maximize yield of a desired product. With conventional fermentors, this can be achieved either by in situ monitoring of the fermentor or by removing (continuously or at frequent time points) sterile samples of the contents and subjecting them to analysis.

In order to gain direct information about the concentration of single compounds in media that usually contain a complex mixture of components, analytical devices that exhibit high-selectivity for target molecules are typically required. To date, this has only been achieved by the employment of various on-line chromatographic procedures, such as liquid chromatography, gas chromatography, and mass spectrometry, and has allowed the simultaneous detection of several compounds. These types of processes, however, require expensive multi-channel devices that can take from 30-60 minutes to analyze a particular set of compounds.

In preferred embodiments of the invention at least one analytical sensor is integrated into the microfermentor. An integrated analytical sensor is a sensor that allows monitoring (which may include detection and/or measurement) of a variable of interest (e.g., an analyte) within the microfermentor vessel without the need to remove a sample of the vessel contents. The parameter of interest may be, but is not limited to: biomass, pH, dissolved oxygen, dissolved carbon dioxide, glucose, lactate, ammonia, ions such as phosphate or metal ions, any cell metabolite (which may be a protein, nucleic acid, carbohydrate, lipid, etc.), temperature. In certain embodiments of the invention the analytical sensor detects and/or measures a cell product that is to be harvested from the microfermentor or a compound that is being removed or metabolized by the cells. In certain embodiments of the invention the analytical sensor detects and/or measures a cell product that is a byproduct of metabolism, e.g., a toxic or growth-inhibitory byproduct.

In certain preferred embodiments of the invention one or more optical sensors is employed. Optical sensors have several advantages over other sensor families. They are largely immune to electromagnetic interference and cross-talk, are non-invasive, fast and work at high temperature, and are capable of continuous monitoring of an analyte even in rugged conditions such as human blood serum and fermentation broths. In addition, another desirable feature of optical sensing (e.g., using optical chemical sensors) is that it generally does not interfere with the process being measured. Furthermore, the materials are usually inexpensive, allowing their incorporation into disposable microfermentors.

In general, an optical sensor is a device that works by detecting, e.g., measuring, induced changes (i.e., changes induced by the presence of an analyte) in the absorptive, luminescent, or fluorescent properties of a medium (the chemical sensor). Generally a system employing an optical sensor includes a light source (i.e., a source of optical excitation) and a means of detecting light. Optical excitation emitted from the source excites an optical chemical sensor, which then emits luminescence or absorbs light. The luminescence emitted from the chemical sensor or the amount of light absorbed by the chemical sensor varies depending upon the concentration of the analyte. Changes in the amount of light emitted or absorbed (measured by the detector) reflect alterations in the concentration of the analyte. The chemical sensor may be supplied in any of a number of different ways. For example, in certain embodiments of the invention the chemical sensor is present in or added to the culture medium. In certain embodiments of the invention the chemical sensor is provided as a component of a sol-gel or polymer matrix or a film, which may coat at least a portion of a vessel wall or may form a structural component of the microfermentor. See, e.g., (67).

Appropriate light sources include, among others, light emitting diodes, lasers, incandescent or fluorescent lights, glow discharge, etc. Appropriate means of detecting light include spectrometers, photodetectors, charge coupled devices, diode arrays, photomultiplier tubes, etc. Optical sensing systems may also include means for collecting light and/or for transmitting it from the source or to the detector, etc. In addition, such systems may include appropriately positioned filters to filter either excitation light or emitted light. In certain embodiments of the invention fiber-optic devices are employed to transmit the light from a source and/or to a detection means. The term “fiber-optic” refers to the medium and the technology associated with the transmission of information as light impulses along a glass or plastic wire or fiber.

In addition to, or instead of, optical sensing systems, any of a wide variety of other technology platforms may be employed. Thus in certain embodiments of the invention chemical or electrochemical sensing systems can be used in conjunction with and/or integrated into the microfermentor. For example, the inventors have shown that infrared photoacoustic spectroscopy scales favorably with miniaturization and can be used as sensitive tool for a wide range of infrared active gases, including CO₂ (11).

A. Oxygen Sensing

1. Integrated oxygen sensor

In certain embodiments of the invention the microfermentor system includes means of monitoring dissolved oxygen (DO) within the vessel. In certain preferred embodiments of the invention an oxygen sensing means is integrated within a structural component of the microfermentor, e.g., within a microfermentor wall (i.e., not separable from the structural component without disrupting the structural integrity of the microfermentor). In certain preferred embodiments of the invention the oxygen sensing means includes an optical sensor. As described in more detail in Example 4 and in (23), oxygen can be detected via fluorescence techniques that exploit the quenching produced by oxygen on fluorophores. Suitable compounds include Ruthenium II tris(4,7-diphenyl-1,1-phenanthroline)₂+. Its fluorescence is quenched in the presence of oxygen, and the relation between dissolved oxygen and fluorescence intensity has been shown to be nearly linear (33). In addition, this compound is sterilizable (34) and has been incorporated into both polymer (34) and sol-gel matrices (35). Such features are desirable for a fluorophore to be used in an optical sensor. Of course any of a number of other oxygen-sensitive compounds may be used. According to certain embodiments of the invention such a compound is incorporated into a structural component of the microfermentor, e.g., into an optically transparent bottom, top, or side wall. For example, as described in more detail in Example 4, the compound may be incorporated into a sol-gel that is applied to a structural component of the microfermentor (in this case a glass slide that forms the microfermentor base). Alternately, the compound may be applied to the bottom, top, and/or one or more sides of the microfermentor interior with or without a support and may be immobilized at this location. The compound may also be incorporated directly into the material from which the structural component is fabricated.

B. pH and Analyte Monitoring

In certain embodiments of the invention the microfermentor system includes means of monitoring the pH of the contents of the microfermentor. In certain embodiments of the invention the microfermentor system includes means of monitoring the presence of one or more analytes in addition to or instead of oxygen. Methods employed in the context of commercially available blood gas (pH, CO₂, O₂) sensors may be adapted for use in the microfermentor. In such sensors pH is detected by a chromophore, which changes its optical spectrum as a function of the pH. Absorption—and fluorescence-based fiber-optic sensors may be used. Carbon dioxide is detected indirectly, since its diffusion in a carbonate solution fixed on the fiber tip alters the pH, so that the carbon dioxide content can be measured by measuring the pH.

Hydrogels, cross-linked networks of hydrophilic polymers, can also be used for pH sensing. These hydrogels swell in the presence of water, and various hydrogels have been synthesized that undergo large changes in their swelling ratio depending on their environment. In addition to pH, responsive hydrogels have been developed that sense various other environmental conditions including temperature, light, electric field, pressure, the presence of carbohydrates, and the presence of antigens. pH-dependent swelling is achieved through the incorporation of weakly basic or acidic groups on the polymer backbone.

Two effects allow the quantification of variable pH-responsive hydrogel swelling. The first effect is the change in optical properties of the hydrogel on swelling. For this purpose a hydrogel membrane, containing embedded microspheres 1 μm in diameter, is synthesized. The membrane is turbid because of the difference in refractive indices between the hydrogel and the microspheres. The turbidity of the membrane decreases in an acidic medium due to the swelling of the microspheres, which lowers their refractive index and brings it closer to that of the hydrogel. The change in turbidity can be detected optically (47).

A second method of quantification involves measuring changes in the hydrogel conductivity. Conductivity changes have been found to reflect differences in ionic mobility within the hydrated gel (48, 49). This effect has been used to microfabricate a conductimetric pH sensor (50, 51). Changes in sensor resistance as large as 45% per pH unit near physiological pH have been reported. Because the sensor operation is based on changes in ion mobility, it operates best in solutions of high ionic strength.

Numerous other methods for performing sensing, e.g., optical sensing, of various analytes are known in the art. See, for example, U.S.S. N. 20020025547; U.S. Pat. Nos. 6,377,721; 6,285,807, and references therein. Other approaches to the use of fiber-optic devices and/or optical chemical sensors are found, for example, in (36-39) and references therein.

C. Temperature Sensing

In certain embodiments of the invention temperature control is achieved by incorporating temperature sensors and resistance heaters into the design as described, for example, in (9). As described therein, the inventors have shown in the context of a micromechanical system that it is possible to heat reaction volumes uniformly while accurately monitoring the temperature. Methods of monitoring temperature using optical chemical sensors are known in the art.

D. Monitoring Biomass

A number of techniques may be employed to detect and quantify biomass (e.g., cell density). In certain embodiments of the invention biomass is monitored using optical density. Sensing of optical density can be carried out using absorbance measurements at 600 nm, as is currently done in laboratory analysis. Absorbance measurements can be made through a transparent portion of the microfermentor vessel wall or using a waveguide. Example 4 describes one embodiment in which a light source provides light to one side of the microfermentor (in this case the bottom), and light transmitted through the microfermentor is captured at a different side (in this case the top). Appropriate light sources, detectors, and light transmission devices are described above. Equipment such as lenses, filters, beam splitters, dichroics, prisms and mirrors may be incorporated to enhance detection and accuracy. According to certain embodiments of the invention a cell that produces an easily monitored reporter enzyme, e.g., a fluorescent or luminescent protein such as green fluorescent protein (GFP) is employed.

The invention also encompasses the detection of cell metabolites including, among others, NAD(P)H (a pyridine nucleotide that is an endogenous chromophore and thus may serve as a fluorescence indicator), as an alternate or complementary means of monitoring biomass (52, 53).

According to certain embodiments of the invention one or more parameters or analytes is measured using Raman spectroscopy (80, 81). This technique may be particularly appropriate for measuring organic compounds, e.g., nutrients, cellular metabolites, etc.

E. Self-Assembling Sensors

On metal surfaces, self-assembly can be used to produce modified electrodes with chemical sensing abilities. For example, thiols will adsorb onto gold microelectrodes patterned on a silicon (oxide) substrate and selectively functionalize the electrodes and not the background substrate (18). The use of electroactive thiol reagents (specifically, a quinone-thiol and a ferrocene-thiol) has provided the ability to generate pH sensors from gold electrodes with a simple fabrication methodology (19). For example, during the microfermentor fabrication, various microelectrodes can be readily introduced strategically into its structure, and self-assembly can be used subsequently to functionalize their surfaces and produce on-board chemical sensors within the device. Present abilities allow the preparation of electrochemical sensors for pH, halide detection, glucose monitoring, and a few other species and can be expanded to provide local probes for other analytes of interest.

F. Enhancing Sensitivity of Sensors

The invention encompasses a variety of approaches to enhance the sensitivity of biosensors by using integrated optical components. One such approach includes the enhancement of the interaction path length for a fluorescent indicator emitting into a waveguide and the absorption path length in evanescent wave spectroscopy. This is realized by the use of planar waveguides in silicon/silicon dioxide. A second approach is to enhance the sensitivity of the fluorescence detection process by integrating silicon avalanche photodiodes with silicon dioxide waveguides. Recently, these avalanche photodiodes have enabled single molecule detection in aqueous flows (21).

1. Waveguide sensors

Fiber optic sensors are only one implementation of what can generally be referred to as waveguide sensors. In general, these sensors rely on the refractive index difference between the waveguide core and the waveguide cladding to confine the light. The optical field, which is present very close to the core surface, is called the evanescent wave and can be used to probe the absorption of the surrounding medium or can be excited by fluorescence. If the cladding is stripped away and the waveguide immersed in a solution of fluorescent indicator, the only fluorescence excited by the light in the waveguide core would come from dye molecules in the sheath surrounding the exposed core. Some of that fluorescence would couple back into the waveguide and come out the ends.

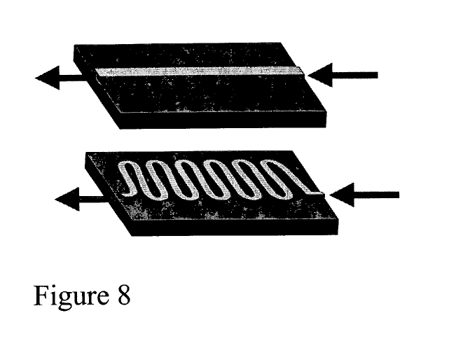

According to certain embodiments of the invention planar waveguides with rectangular cross-section are integrated on a microscale bioreactor platform. These devices allow for dramatic enhancements in interaction path length by virtue of the serpentine paths the waveguide can take through the analyte. For example, a serpentine waveguide can compress a 1 meter optical path length on a one square centimeter surface area (see FIG. 8). More importantly the total volume of this waveguide can be smaller than one nanoliter. As such, the planar waveguide can realize macroscopic optical cross-sections through microscopic analyte volumes. In certain embodiments of the invention the microscale bioreactor incorporating a waveguide sensor has an interior volume of less than or equal to 1 ml. In certain embodiments of the invention the microscale bioreactor incorporating a waveguide sensor has an interior volume of less than 200 μl. In certain preferred embodiments of the invention the working volume is between 50 μl and 100 μl inclusive. In certain preferred embodiments of the invention the working volume is between 5 μl and 50 μl, inclusive. In certain preferred embodiments of the invention the working volume is between 5 μl and 10 μl, inclusive. In certain preferred embodiments of the invention the working volume is approximately 7.5 μl or approximately 10 μl. In certain preferred embodiments of the invention the working volume is approximately 5 μl. Waveguide sensors may be fabricated using any appropriate technique. (See, e.g., U.S. Pat. No. 6,355,198 for some approaches.)

2. Single Photon Avalanche Diodes

The small volumes of the microscale bioreactors necessarily mean that analysis must be performed on small volumes of analyte. While the waveguide biosensor may have maximal interaction with the available analyte, in certain embodiments of the invention further sensitivity is realized by direct integration of photodetectors with the waveguides. Recent advances in single molecule detection within a flow cell have been made possible by the development of a single-photon avalanche diode (SPAD) with high quantum efficiency and low timing jitter. The increased fluorescence detection efficiency provided by the SPAD has enabled the detection of single chromophore molecules (23).

Silicon avalanche photodiodes with 90% quantum efficiency for wavelengths from 400-800 nm are commercially available. These devices have an internal electrical gain of 40-100 due to the avalanche process and exhibit very low noise as well as high dynamic range. Microfabricated SPAD can be easily integrated with waveguide biosensors. In this way fluorescence can be monitored from even a small number of molecules for virtually all visible and near-infrared markers used in biochemistry.

3. Optical background in bioreactors

A significant obstacle to coupling an optical sensor to the fermentation process is interference from the medium broth. This is due to the content of the fermentation broth, which contains cells and other opaque components. These materials absorb and scatter light, which interferes with the optical signal. The invention encompasses three approaches to deal with the complexities of bioprocess monitoring.

The first is to integrate microporous filters along the sensing surface of the waveguides. Recently, waveguide based optical sensors based on immobilization of a ruthenium complex in Nafion to monitor pH in a fermentation of Klebsiella pneumoniae have been demonstrated. Interference from the culture medium was eliminated by the addition of a black microporous filter membrane on top of the sensing film (24). These filter membranes can either be deposited after waveguide processing or they can be directly microfabricated during the sensor process.

A second approach is to employ high speed SPAD for fluorescence-lifetime spectroscopy. It has been well documented that fluorescence-lifetime methods can be successfully applied in optical sensing. These methods have considerable advantages over intensity-based methods. The fluorescence lifetime of an indicator is an intrinsic property and is virtually independent of fluctuations in light-source intensity, detector sensitivity, light throughput of the optical system, sensing layer thickness and indicator concentration (25). This implies that, in contrast to absorption methods, no reference measurement system is necessary, and, in contrast to fluorescence-intensity measurements, no compensation for variation of instrumental parameters is necessary. Lifetime-based sensors can be stable over years without any need for recalibration (26).

G. Multiple Sensing Means

Regardless of the sensing methodology employed, in certain embodiments of the invention the microscale bioreactor incorporates multiple sensors (e.g., at least 2, 3, 4, 5, or even more), thus allowing monitoring of multiple bioprocess parameters. In certain embodiments of the invention the microfermentor incorporates a sensor for monitoring oxygen. In certain embodiments of the invention the microfermentor incorporates sensors for monitoring oxygen and at least one other analyte or parameter. In certain embodiments of the invention the microfermentor incorporates sensors for monitoring oxygen and pH. In certain embodiments of the invention the microfermentor incorporates sensors for monitoring oxygen, temperature, and at least one other analyte or parameter. The sensors may be based on the same technology platform (e.g., the sensors may all be optical chemical sensors) or may be based on different technology platforms. In certain embodiments of the invention biomass and at least one additional parameter (e.g., dissolved oxygen concentration) are monitored optically. In certain embodiments of the invention the additional parameter is monitored using an optical chemical sensor. Monitoring may take place continuously, and multiple parameters may be monitored simultaneously. Where optical sensors are used it is important to avoid confounding of sensors where possible. For example, it may be important to account for the fact that absorbance readings for optical density measurements are typically made at 600 nm.

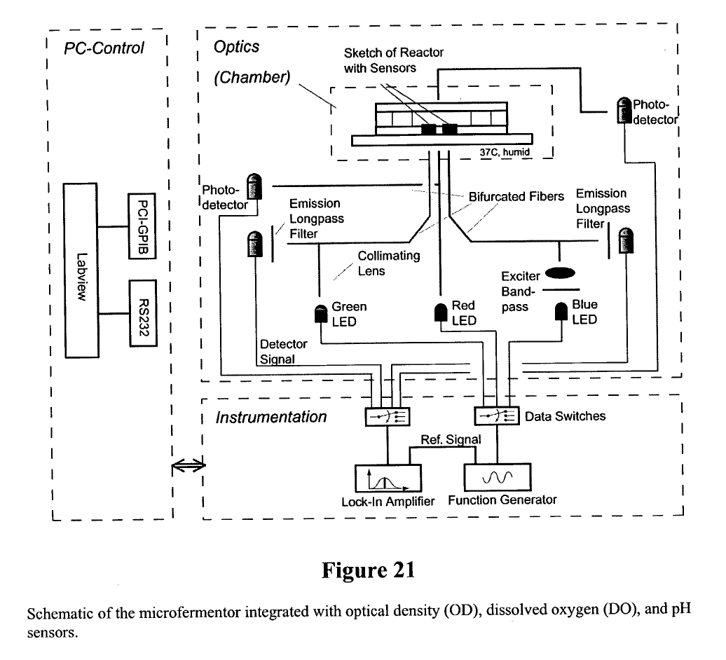

The information obtained by monitoring may be used to control and/or alter microfermentor conditions. Such monitoring and alteration may be controlled by appropriate software (e.g., the LabView system). In the case of a microfermentor array, each microfermentor may be monitored and controlled individually. FIG. 21 shows a schematic of a microfermentor integrated with optical density, dissolved oxygen, and pH sensors. As shown on FIG. 21, the microfermentor and associated optics interfaces with instrumentation and computer software to measure and/or control bioprocess parameters (see below).

V. Bioprocess Parameter Control

As described herein, in addition to monitoring of bioprocess parameters, in certain embodiments of the invention one or more of these parameters may be actively controlled and/or varied.

A. Gas Exchange

In certain embodiments of the invention oxygen delivery and/or removal of waste gases such as carbon dioxide is accomplished via a gas-permeable membrane. Preferably such a membrane is relatively impermeable to the components of the culture medium. In general, two categories of membranes that are typically used to aerate cultures—open-pore membranes (e.g. polypropylene (PP) and polytetrafluoroethylene (PTFE)), and diffusion membranes (e.g. PDMS), may be used to aerate the microfermentor.

Porous membranes consist of a polymeric matrix that contains pores from 2 nm to 10 μm in diameter. Many pore geometries exist, and together with the wide range of pore sizes give rise to several different regimes of O₂ transport, including Knudsen diffusion (narrow pores) and viscous flow (wide pores) (59). Mass transfer through a diffusion membrane (which contains molecular pores) is a function of a thermodynamic parameter, the solubility S, and a kinetic parameter, the diffusivity D. Which of these parameters dominates the mass transfer for a given polymer and penetrant depends on the nature of the interaction between the two.

Suitable materials for membranes include, for example, fluoropolymers such as the microporous membranes Teflon (e.g., Teflon AF 2400, DuPont), Goretex, cellulose acetate, porous glasses (e.g., Vycor), microporous ceramic membranes (e.g., made by sol-gel techniques), zeolite membranes, and silicones such as the diffusion membrane PDMS. Relevant permeability, solubility, and diffusivity parameters of PDMS and Teflon AF2400 are presented in Tables 1, 2, and 3 (data from 60-66).

TABLE 1
Summary of Gas Permeability, Solubility, and
Diffusivity Parameters in PDMS at 35° C.

| | P × 10¹⁰ [cm³(STP) · | S [cm³(STP)/ | D × 10⁵ |
|---|---|---|---|
| Penetrant | cm/cm² · s · cmHg)] | cm³polymer · atm] | [cm²/s] |
| O₂ | 800-933 | 0.18 | 3.4 |
| CO₂ | 3800-4570 | 1.29-1.31 | 2.2-2.64 |

TABLE 2
Summary of Water Permeability, Solubility, and Diffusivity
Parameters in PDMS at 300K

| | P₁ × 10⁹ | P_{g} × 10⁵ | | | |
|---|---|---|---|---|---|
| Penetrant | [cm²/s] | [cm²/s] | S₁ × 10³ | S_{g} | D × 10⁵ [cm²/s] |
| H₂O | 4.2-10.0 | 9.1 | 0.276-1.0 | 5.9 | 1.53-2.0 |

TABLE 3
Summary of Gas Permeability in Teflon AF 2400 at 25° C.

| Penetrant | P × 10¹⁰ [cm³(STP) · cm/cm² · s · cmHg)] |
|---|---|
| O₂ | 1600 |
| CO₂ | 3900 |

In Table 2, the solubility S is defined as the ratio of the number densities between two phases and is used to calculate the concentration at the polymer interface given the concentration in the bulk solution on both sides of the membrane. The permeability P then has units of diffusivity D, and can be thought of as an “adjusted” diffusivity. This is in contrast to the units that are normally given to permeability (Table 1), arising from the relations:

P=DS

and

$$N = \frac{D}{t} ( {C}_{1} - {C}_{2} )$$

where N is the penetrant flux through the membrane. One of ordinary skill in the art will be able to select membrane materials having appropriate diffusivities and solubilities for water, oxygen, carbon dioxide, and other penetrants.

Preferred materials are biocompatible, relatively strong, and capable of being formed into thin membranes (e.g., membranes with thicknesses on the order of the dimensions of the microfermentor. The external face of the membrane (i.e., the face not in contact with the contents of the microfermentor) is in contact with a source of oxygen that has a higher oxygen concentration than the concentration of oxygen in the microfermentor culture vessel. This oxygen source may be a gas or a liquid. In certain embodiments of the invention the source is a gas with a higher oxygen content than air. Oxygen diffuses across the membrane to provide oxygenation for the cells within the microfermentor. In certain embodiments of the invention two or more separate membranes are incorporated into the microfermentor. The external surface of the second membrane may be in contact with a gas or liquid having a lower oxygen content than the contents of the microfermentor vessel. In this manner an oxygen gradient is established across the microfermentor vessel, which facilitates oxygenation. By varying the relative oxygen concentrations with which the external faces of the membranes are in contact, it is possible to control the oxygen concentration within the microfermentor.

Although aeration membrane(s) are employed in preferred embodiments of the microfermentor system, the invention also encompasses the use of other means of providing oxygen, e.g., miniaturized magnetic stirrers, bubbling action of aeration, piezoelectric vibration, or chemical production of oxygen (in which case it is desirable to avoid the formation of toxic byproducts).

In preferred embodiments of the invention sufficient oxygen is provided to the interior of the microfermentor to support the viability and growth of bacterial cells undergoing aerobic metabolism at cell densities comparable to those employed in standard fermentation processes (e.g., approximately 10¹² cells/liter). In certain embodiments of the invention sufficient oxygen is provided to support exponential growth of bacterial cells undergoing aerobic metabolism at a range of cell concentrations, e.g., at up to approximately 10⁶ cells/l, up to approximately 10⁷ cells/l, up to approximately 10⁸ cells/l, up to approximately 10⁹ cells/l, up to approximately 10¹⁰ cells/l, up to approximately 10¹¹ cells/l, up to approximately 10¹² cells/l, or up to approximately 10¹³ cells/l. As is well known in the art, mammalian cells typically have a lower oxygen uptake rate than aerobic bacteria.

B. Climate Control

1. Temperature Control

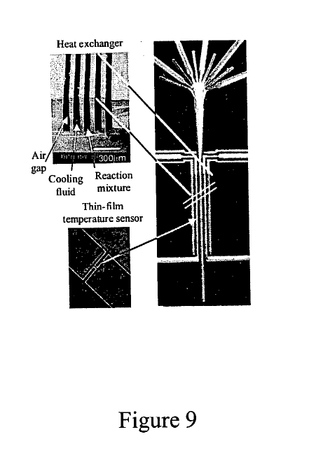

As mentioned above, in certain embodiments of the invention temperature control is achieved by incorporating temperature sensors and resistance heaters into the design of the microfermentor. For example, the inventors have shown in the context of a micromechanical system that it is possible to heat reaction volumes uniformly while accurately monitoring the temperature (9). In addition, in certain embodiments of the invention heat exchangers for heating and cooling are incorporated into the microfermentor in a fashion analogous to that described in (10). An example of a microfabricated heat exchanger is shown in FIG. 9. The excellent heat transfer characteristics of small dimension microfabricated devices provide good thermal uniformity and small time constants. In certain embodiments of the invention the temperature is controlled to within ±2° C. In certain embodiments of the invention the temperature is controlled to within ±1° C. In certain embodiments of the invention the temperature is controlled to within ±0.1° C.

In certain embodiments of the invention temperature control is achieved by placing the microfermentor in a temperature-controlled environment, for example by placing the microfermentor in a temperature-controlled incubator or chamber as described in Example 3. Temperature control can be achieved, for example, by flowing water of a desired temperature through a chamber base.

2. Evaporation Control

In certain embodiments of the invention an appropriate humidity is maintained by placing the microfermentor in a humidity-controlled environment. For example, as described in Example 3, the microfermentor may be placed in a chamber that contains open reservoirs of water. Alternatively, humidified air may be flowed through the chamber. In preferred embodiments of the invention the chamber is sealed. Sealing the channels that lead into the microfermentor also minimizes evaporation. In addition, appropriate selection of materials for the structural components of the microfermentor (e.g., selection of hydrophobic materials) reduces evaporation.

In certain embodiments of the invention one or more membranes, one side of which in contact with the interior of the microfermentor vessel and the other side of which is in contact with humidified air or water, compensates at least in part for evaporative losses. The humidified air or water may be flowed past the membrane. As described above, various designs incorporating two vessels separated by a gas-permeable membrane may be employed.

C. pH Control

In large part because protein configuration and activity are pH dependent, cellular transport processes, reactions, and hence growth rates depend on pH. Factors such as ongoing metabolic activity may alter the pH in a culture medium. Therefore, certain embodiments of the invention include a means to control the pH. In certain embodiments of the invention pH control is achieved by providing a suitable buffer. The buffer may be provided within the culture medium. Alternately, an external buffer source may be employed, in which case the invention includes a contact between the external buffer source and the interior of the microfermentor vessel. For many bacteria, growth rates typically reach a maximum in the pH range of 6.5-7.5 (55). Typically, negligible growth occurs at a pH 1.5 to 2.0 pH units above or below the optimal pH. Many eukaryotic cells are even more sensitive to changes in pH. Accordingly, in certain embodiments of the invention the microfermentor system includes a means of controlling the pH within ±1 pH units of an optimum pH for cell growth. In certain embodiments of the invention the microfermentor system includes a means of controlling the pH within ±2 pH units of an optimum pH for cell growth. In certain embodiments of the invention the microfermentor system includes a means of controlling the pH within ±5 pH units of an optimum pH for cell growth. In certain embodiments of the invention the microfermentor system includes a means of controlling the pH within ±1 pH units of an optimum pH for cell growth. In certain embodiments of the invention the microfermentor system includes a means of controlling the pH within ±1.5 pH units of an optimum pH for cell growth. In certain embodiments of the invention the microfermentor system includes a means of controlling the pH within ±2 pH units of an optimum pH for cell growth. One of ordinary skill in the art will readily be able to determine the optimum pH for cell growth by reference to the scientific literature and/or by systematically culturing cells under conditions of varying pH while holding other parameters constant. The optimum pH may vary depending upon other culture parameters, e.g., nutrient supply, temperature, etc.

D. Nutrient Control

According to certain embodiments of the invention addition of nutrients, stimulants, buffers, etc., is achieved through the use of external pressure driven flows, e.g., created by pumps such as syringe pumps. See also (40) and references therein. When possible, active fluid control elements may be used. Development of such elements, e.g., valves, is currently under way in the microelectromechanical systems community and will readily be applicable in the context of the microfermentors described herein.

Alternatively, nutrients may be provided by diffusion through a membrane, e.g., from a larger reservoir, so that components are constantly renewed. Certain of the two-vessel designs described above allow for this feature.

E. Agitation

In certain embodiments of the invention agitation is used to assist in keeping the cells in suspension and prevent them from settling on the bottom of the microfermentor. Liquid within the microfermentor may be agitated by attaching the microfermentor to a moving surface (as is the case with shake flask agitation). Alternative methods of agitation may also be employed, e.g., piezoelectric effects, stirring with magnetic beads, etc.

F. Bioprocess Control in Microfermentor Arrays

The invention provides microfermentor systems comprising a plurality of microfermentors in which one or more bioprocess parameters is controlled. An exemplary embodiment is depicted in FIG. 4B. According to certain embodiments of the invention the system comprises individually addressable wells, whereby each well may receive a unique combination of inputs. According to certain embodiments of the invention each well receives the same input along one dimension and a different input along a second dimension of the array. This approach is not limited to two dimensions; rather any number of different inputs may be provided. According to certain embodiments of the invention the microfermentors are accessed by microfluidic channels. The wells may be housed in a plate or platform comprising multiple layers, one or more of which may contain channels that connect to the wells. The wells may also be addressed electronically, e.g., via wires extending therefrom. Electronic addressing may be used to control components within the wells. For example, electronic addressing may be used to control resistors within the wells to regulate temperature. In addition, data may be gathered from each well independently.

VI. Methods of Using Microfermentors and Microfermentor Arrays

A. Introduction

Fermentations are important sources of biological products used in the pharmaceutical, food, and chemical industries (54, 68-73). These products include primary and secondary metabolites, enzymes, recombinant proteins, vaccines, and the cells themselves (e.g., yeast). A hallmark of commercial fermentation processes (e.g., processes performed in production scale fermentors, by which is meant fermentors with working volumes of between 10 and 300,000 liters) has been an attempt to promote enhanced production of these industrial products through improvement of strains and/or optimization of fermentation conditions.

Strain improvement has typically been achieved through one of several procedures (mutation, genetic recombination, and genetic engineering), all of which bring about changes in the DNA sequence. These techniques are frequently used in combination with each other to reach the desired goal. Currently, improved strains are selected using an iterative cycle of three basic principles: mutation, screening, and assay. Manual screening operations are typically carried out in shake flasks or test tubes. Mutants are cultured in a primary screen, and hits are identified by measuring the total product yield using an assay such as thin layer chromatography (TLC), high-performance liquid chromatography (HPLC), or the increasingly popular enzyme-linked immunosorbent assay (ELISA). Identified hits are then taken forward and run through additional screens for confirmation.

Additionally, fermentation and cell culture can play a critical role in the elucidation of gene function in other organisms. The most common method involves the cloning and expression of a genome in a suitable host, such as E. Coli or yeast, followed by fermentation in a bioreactor. The fermentation allows the identification of conditions that regulate gene expression, as well as production optimization of the protein that is then expressed. Complete genomic sequences are currently available for a wide variety of organisms including bacteria, fungi, and plants, and the amount of genomic sequence data is growing rapidly. (See, e.g., sequences available at the Web site having URL www.ncbi.nlm.nih.gov/entrez/query.fcgi?db=Genome) In particular, the recent completion of the human genome sequence provides an especially labour-intensive challenge in this area. The same issues that were identified above for the screening of improved strains are of concern here, and here again the opportunity exists for the miniaturization of culture conditions.

B. Cell Types

The microscale bioreactors of the invention may be used to culture and monitor cells of any type including microorganisms such as bacteria (e.g., eubacteria, archaebacteria), filamentous or non-filamentous fungi (e.g., yeast), protozoa, and also plant cells, insect cells, mammalian cells, etc. Bacteria may be aerobes, facultative anaerobes, or anaerobes and include, but are not limited to, members of the following genera: Escherichia, Enterobacter, Streptomyces, Azotobacter, Erwinia, Bacillus, Pseudomonas, Klebsiella, Proteus, Salmonella, Serratia, Shigella, Rhizobia, Rhodococcus, Vitreoscilla, and Paracoccus. (See the Web sites with URLs www.bacterio.cict.fr/cubacteria.html and www.bacterio.cict.fr/archaea.html for lists of bacteria that may be used.). Yeast include, but are not limited to, members of the genera: Saccharomyces, Schizosaccharomyces, Moniliella, Aureobasidium, Torulopsis, Candida, Trigonopsis, Trichosporon, Torulopsis, Zygosaccharomyces, and Yallowia. Insect cells, e.g., cells that support the growth of baculovirus such as Spodoptera frugiperda sf9 cells (see, U.S. Pat. No. 4,745,051) may be used. Such cells are particularly useful for production of recombinant proteins. Mammalian cells including, but not limited to, Chinese hamster ovary (CHO) cells, human embryonic kidney (HEK) cells, COS cells etc., may be used. See (76). In certain preferred embodiments of the methods described below the cells are of a type that is currently used in commercial bioprocesses.

The cells may be newly isolated or identified naturally occurring strains or variants, which may also be referred to as mutants. The cells may be selected, e.g., for a desirable phenotype. The cells may be genetically modified, e.g., using recombinant DNA technology. For example, cell or strain variants or mutants may be prepared by introducing appropriate nucleotide changes into the organism's DNA. The changes may include, for example, deletions, insertions, or substitutions of, nucleotides within a nucleic acid sequence of interest. The changes may also include introduction of a DNA sequence that is not naturally found in the strain or cell type. One of ordinary skill in the art will readily be able to select an appropriate method depending upon the particular cell type being modified. Methods for introducing such changes are well known in the art and include, for example, oligonucleotide-mediated mutagenesis, transposon mutagenesis, phage transduction, transformation, random mutagenesis (which may be induced by exposure to mutagenic compounds, radiation such as X-rays, UV light, etc.), PCR-mediated mutagenesis, DNA transfection, electroporation, etc.

The complete genomic sequence is available for a number of different organisms including numerous bacterial species. The availability of the genomic sequence has facilitated the construction of panels of mutants, each of which bears a loss-of-function mutation in one or more genes or open reading frames (42). In some cases the particular gene bearing the loss-of-function mutation is “tagged”, making it possible to identify a particular mutant in a mixed population.

One of ordinary skill in the art will be able to select appropriate culture media and environmental conditions for any particular cell type. Parameters such as oxygen delivery, temperature, and pH, etc., may be varied as appropriate. In addition, the microfermentor properties such as surface characteristics, vessel size, etc., may be modified depending upon the features of the particular cell type to be cultured.

B. Screening for Optimal Strains

The microscale bioreactors of the invention may be used to identify optimal organisms for performing a bioprocess. Since the microfermentors allow multiple fermentations to be performed in parallel under similar or identical conditions, they find particular use in selecting a cell type that performs optimally under such conditions, e.g., a cell type that produces a maximum amount of a desired product, a cell type that does not require a particular nutrient, etc.). The similar or identical conditions may include, but are not limited to: growth medium (carbon source, nitrogen source, precursors, and nutrients such as vitamins and minerals, salts, etc.), temperature, pH, redox potential, agitation rate, aeration rate, ionic strength, osmotic pressure, water activity, hydrostatic pressure, dissolved oxygen or carbon dioxide concentration, concentration of inducers and repressors, etc. The microfermentors are useful in screening panels of naturally occurring strains, banks of mutants, banks of genetically modified organisms, etc. Multiple different cell types or strains may be cultured in parallel under similar or identical conditions. The same cell type may be grown at a range of different cell densities. Strains, mutants or variants of particular interest include, but are not limited to, auxotrophic strains, deregulated mutants, mutants resistant to feedback inhibition, mutants resistant to repression, etc. See (68) for further discussion.

An optimum strain may be selected based on a variety of criteria. For example, an optimum strain may be, but is not limited to: a strain that produces the greatest amount of a desired product in a given time; a strain that is able to produce a desired product using a particular starting material (e.g., an inexpensive starting material); a strain which is able to grow in medium lacking particular components; a strain that is able to tolerate buildup of toxic or inhibitory metabolites in the culture; a strain that is able to tolerate a wider range of growth conditions such as pH, oxygen concentration, etc.; a strain that is able to achieve a higher cell density, etc.

C. Optimizing Bioprocess Parameters

The microscale bioreactors of the invention are useful in identifying optimal bioprocess parameters for performing a given bioprocess. Since the microfermentors allow control and/or monitoring of multiple variables, e.g., biomass, oxygen concentration, etc., they may be used to determine what values for these variables lead to optimum production of a desired metabolite or optimum removal of an undesired compound. For example, the maximum growth rate may not be the optimal growth rate for such purposes. Growing cells at less than the maximum growth rate may help minimize the accumulation of byproducts that negatively impact the growth or metabolism of the organism.

Parameters that may be varied include, but are not limited to: growth medium (carbon/energy source (e.g., glycerol, succinate, lactate, and sugars such as, e.g., glucose, lactose, sucrose, and fructose), nitrogen source, precursors, and nutrients such as vitamins and minerals, salts, etc.), temperature, pH, redox potential, agitation rate, aeration rate, ionic strength, osmotic pressure, water activity, hydrostatic pressure, dissolved oxygen or carbon dioxide concentration, concentration of inducers and repressors, etc. Any of these parameters may be varied in different ways in individual microfermentors operating in parallel, so that a time-optimal manner of varying the parameters can be identified, e.g., a manner of varying the parameters so as to optimize the process, e.g., to maximize production of a desired metabolite or maximize removal of an undesired compound. See (68) for further discussion.

The availability of a large number of microfermentors, e.g., as a microfermentor array, makes it possible to systematically vary a single parameter across a wide range of values while holding other parameters constant. Perhaps of greater significance, the availability of a large number of microfermentors makes it possible to assess the effects of simultaneously varying multiple parameters across a range of values. Appropriate mathematical techniques (which will likely be embodied in software) may be employed to determine which of these parameters is significant in terms of effects on a desired output, e.g., product level or removal of an undesired compound from the culture medium See 68 and references therein, describing use of software packages such as JMP (SAS, Cary, N.C., USA) and use of experimental designs such as Plackett-Burman screening design, fractional factorial design, response surface methodology, Box-Wilson central composite design, etc. Multiple microfermentors may be operated under each set of bioprocess parameters, which may greatly increase the reliability and statistical significance of the data.

Once one or more cell strains and/or bioprocess parameters is selected using the microscale bioreactors, scale-up (e.g., to production scale fermentors) may be performed. In performing scale-up, the skilled artisan will take into account factors such as differences in oxygenation technique between microfermentors and production scale fermentors, different geometries, different shear stresses, etc. (See 68, 74, 75).

D. Additional Applications

The microfermentors and microfermentor arrays also find use in screening compounds to determine their effects on cells. For example, they may be used to identify compounds that inhibit or reduce the growth of cells and/or exert other deleterious effects on cells (e.g., DNA damage). Screening for potential deleterious effects on cells is a necessary step in the testing and/or development of compounds for any of a wide variety of uses in which plants, animals, and/or humans will be exposed to the compound. In addition, compounds that reduce or inhibit cell viability and/or growth may be useful as pharmaceuticals, disinfectants, etc. The microfermentors and microfermentor arrays may also be used to identify compounds that increase or enhance the growth of cells, that increase the ability of the cells to produce a desired metabolite or remove an undesired product, etc.

The invention encompasses the use of the microfermentors and microfermentor arrays to determine the response of cells to a compound. A “response” includes, but is not limited to a change in a parameter such as: viability, growth rate, production of a metabolite or other biosynthetic product, biotransformation of a compound, transcription of a gene, expression of a protein, etc. In general, the methods for using the microfermentors and microfermentor arrays include culturing a cell in the presence of a compound of interest and comparing the value of a parameter of interest in the presence of the compound with the value of the parameter in the absence of the compound or in the presence of a different concentration of the compound.

VII. Evaluation of Microfermentors and Comparison with Conventional Fermentor Technology

In certain embodiments of the invention results in the microfermentor reliably predict results that would be obtained by scaling up a bioprocess, e.g., to the scale of a commercially available fermentor. For example, in certain embodiments of the invention a strain that is identified as an optimum strain when cultured in a microfermentor is also an optimum strain when cultured under substantially the same conditions in a conventional fermentor. In certain embodiments of the invention conditions that lead to maximum production of a biosynthetic product or metabolite or that lead to maximum biotransformation or removal of an undesired compound when cells of a particular type are cultured in a microfermentor also lead to maximum production of a biosynthetic product or metabolite or to maximum biotransformation or removal of an undesired compound when cells of the same type are cultured in a conventional fermentor, e.g., a bench-scale fermentor having a culture vessel having a volume of at least 0.5 liters, or a production scale fermentor, which may have a volume of hundreds or thousands of liters. However, it is not necessary that optimum conditions in a microfermentor correspond exactly to optimum conditions in a conventional fermentor, or that rates (e.g., rates of production or removal of a compound, rates of nutrient flux, rates of gas or heat transport, etc.) under a given set of conditions correspond exactly to rates that would be obtained under substantially identical conditions in a conventional fermentor. Rather, in certain embodiments of the invention it is sufficient if conditions and/or rates obtained when cells are grown in a microfermentor may be used to predict behavior when the process is scaled up.

For purposes of initially determining how conditions in a microscale bioreactor correspond or translate to conditions in a larger scale bioreactor, it is desirable to employ a cell type or strain that is well characterized, e.g., in terms of its physiology and behavior under different conditions. Escherichia coli represents an attractive prokaryotic cell choice for use in analyzing microscale bioreactor performance and scale-up. There is a large body of literature describing the physiology of this organism (see, e.g., 41) and its behavior under different reactor conditions. In addition, this organism is currently used in a range of commercial processes including production of small molecules and screening of gene libraries. The chemical composition of this organism is very well understood in terms of elemental composition and major biochemical fluxes. Finally, this organism has been extensively studied at the genetic level; vast collections of mutants are available with many useful properties, and the complete genomic sequence of this species has been determined. A comparable degree of information on the budding yeast Saccharomyces cerevisiae is available, making this an attractive eukaryotic cell type for use in analyzing microscale bioreactor performance and scale-up.

In a number of organisms, various promoters are known to respond to different environmental conditions such as temperature, ion concentration, oxygen concentration, etc., or to physiological insults such as DNA damage, oxidative stress, etc, by increasing or decreasing transcription from a linked gene. In order to determine whether bacteria being cultured in a microfermentor are experiencing physiological stress, and in order to compare growth properties in the microfermentor with growth properties in a larger scale fermentor, strains bearing reporter genes in which such a promoter controls expression of a reporter gene (e.g., luciferase) may be employed.

Various modifications and variations of the invention described herein will be evident to one of ordinary skill in the art and are also within the scope of the claims.

### EXAMPLES

### Example 1

### Fabrication of a Microscale Bioreactor

Poly(dimethylsiloxane) (PDMS) was selected as the microfermentor fabrication material in part because of its biocompatibility and optical transparency in the visible range. The high gas permeability of this material also allows it to be used as the material for an aeration membrane. Glass was selected as the microfermentor base for its transparency and rigidity.

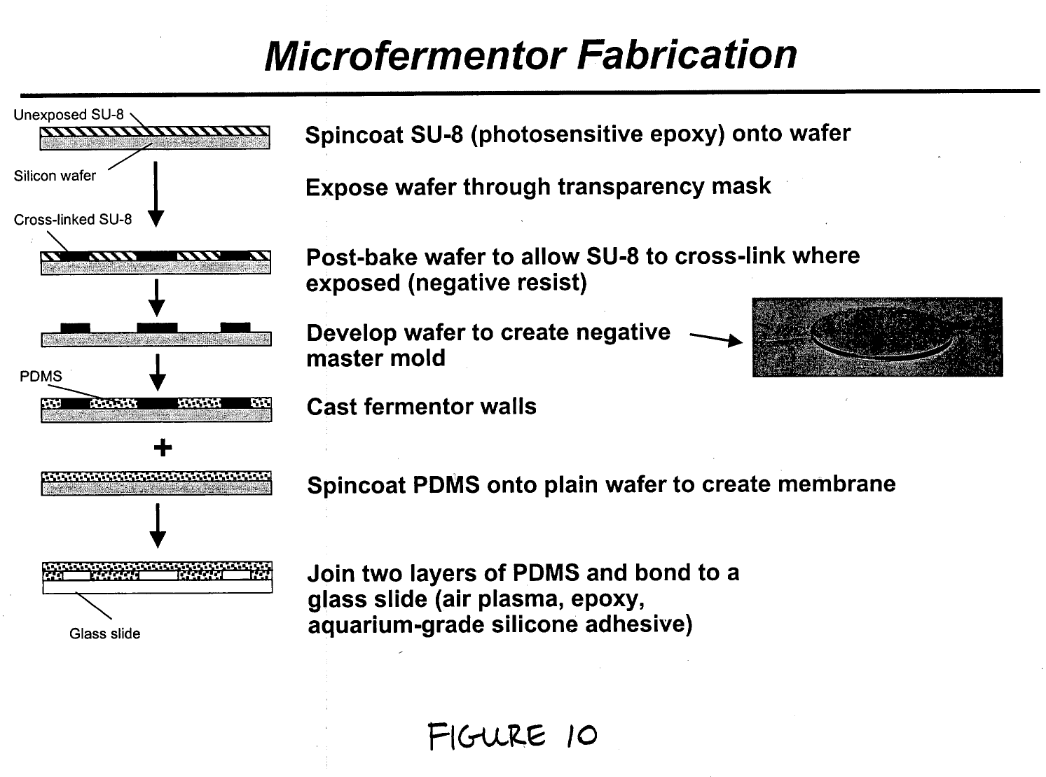

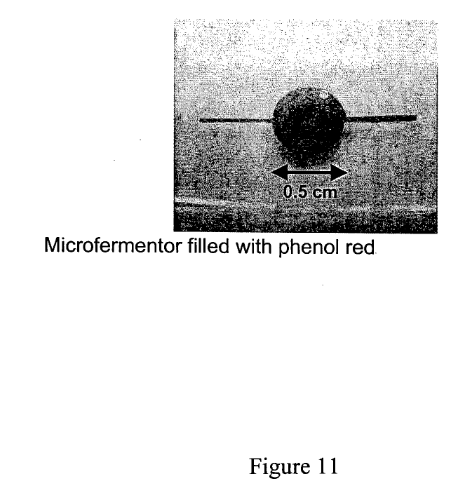

The fabrication procedure used is depicted in FIG. 10. Fabrication of the microfermentor was carried out using soft lithography as described in (58). In the first step of the fabrication process photolithography was used to fabricate a negative master out of silicon and the photo-definable epoxy SU-8. The body of the microfermentor was then cast in PDMS by squeezing the liquid polymer between the negative master and a piece of cured and passivated (silanized) PDMS. The aeration membrane was made by spin-coating the liquid polymer onto a blank wafer. The body and the membrane were subsequently joined and attached to a glass slide using epoxy or other suitable adhesives (e.g., silicone adhesives). (An air plasma seal was initially used to join the membrane to the fermentor body. However, this method appeared to result in a higher rate of evaporation of microfermentor contents, possibly due to the creation of SiO⁻groups on the surface of the PDMS that render the surface hydrophilic. Evaporation can be avoided by, for example, maintaining the microfermentor in a humidified chamber.) A top view of a completed microfermentor filled with phenol red is shown in FIG. 11. The microfermentor has a diameter of approximately 5 mm and a depth of approximately 300 μm. The working volume of the microfermentor vessel is approximately 5 μl. Channels with a 300 μm×300 μm square cross-section extend outwards from and communicate with the vessel interior.

### Example 2

### Modeling Aeration Within a Microscale Bioreactor

Modeling of oxygen diffusion into the microfermentor was carried out using a one-dimensional resistance-in-series model of the membrane and the medium, taking oxygen consumption to be a zeroth-order reaction term (constant oxygen consumption/viable cell). For calculations at 35° C., an oxygen diffusivity in PDMS of 3.4×10⁻⁵ cm²/s and a solubility of 0.18 cm³ (STP)/cm³/atm were assumed (44). For oxygen in water a diffusivity of 2.5×10⁻⁵ cm²/s and a solubility of 7 mg/l were used (45), and it is assumed that values for culture medium would be approximately the same. A typical E. coli oxygen uptake rate (OUR) of 30 (mmol O₂)/(gram dry cell weight/h) was assumed (46).

The models assumed a stagnant medium (no mixing). If some method of mixing is implemented, the maximum depth of the microfermentor will increase. The model assumes steady state conditions (see below for transient analysis of oxygen transport during growth). For the case where cells are spread uniformly throughout the microfermentor volume (homogeneous case), the following equations were obtained:

$${C}_{r} - {C}_{o} = {R}_{V} [ \frac{t d}{{D}_{PDMS}} + \frac{{d}^{2}}{2 {D}_{{H}_{2} O}} ]$$

Where: Rv is the volumetric consumption term

D is the diffusivity of oxygen in PDMS and H₂O, respectively

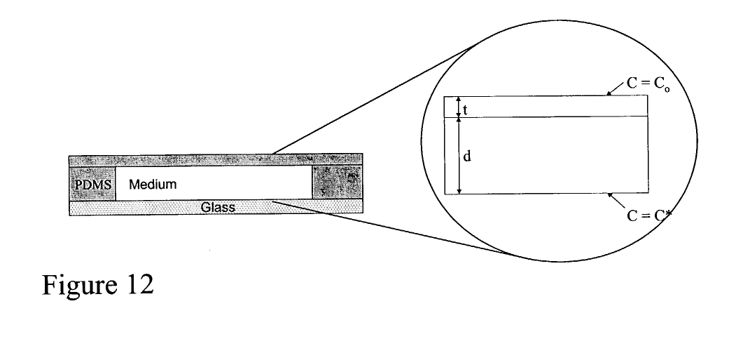

Cᵣ (C* in FIG. 12) is the critical oxygen concentration below which bacteria turn on anaerobic metabolic pathways (Cr=0.0082 mmol O₂/L) (from 55)

Because the solubility of oxygen in water is the main limitation (and not the permeability of the PDMS membrane) the model can be simplified by considering the medium only.

$$C ( x ) = {C}_{o} + \frac{{R}_{V} d}{D} x - \frac{{R}_{V}}{2 D} {x}^{2}$$

In the equation above C is the concentration at x, and x is the axis along the microfermentor depth.

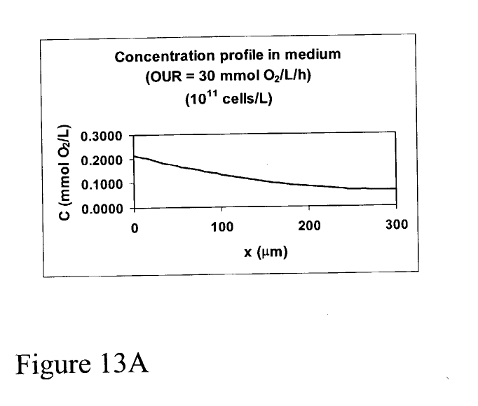

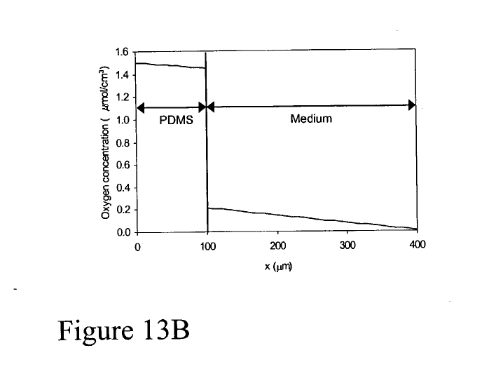

The resulting plot of the oxygen concentration profile within the medium is shown in FIG. 13A.

For the case in which all cells are at the bottom of the microfermentor and consumption is heterogeneous (boundary condition), the following diffusion equation applies:

$${C}_{o} - {C}_{r} = F [ \frac{t }{{D}_{PDMS}} + \frac{d}{ {D}_{{H}_{2} O}} ]$$

Here F is the flux of oxygen at the bottom of the microfermentor, corresponding to the oxygen consumption per unit area. This is converted to a volumetric term by multiplying by the ratio (A/V).

As in the homogeneous case discussed above, the maximum flux will not be realized because the limiting factor is again the solubility of oxygen in water. This can be FIG. 13B, which shows an oxygen concentration profile in the PDMS and the medium itself The assumptions for this figure are again a cell population of approximately 10¹¹ cells/L, and a corresponding OUR of 30 mmol O₂/L/h. A membrane thickness of 100 μm, and a microfermentor depth of 300 μm were used.

As shown in FIG. 13B, the diffusion process is limited primarily by the low solubility of oxygen in water, as evidenced by the large drop-off in oxygen concentration between the membrane and the water. The diffusivity of oxygen in both phases is high enough that the slope of the profile in each phase is relatively shallow. In this case the high oxygen diffusivity combined with a high solubility in PDMS suggested that similar results would have been achieved using a thinner membrane.

The model indicates that due to the high solubility of oxygen in PDMS, the diffusivity of oxygen through the membrane could be up to an order of magnitude smaller and still provide adequate oxygenation. Therefore, any membrane with a high oxygen solubility would be compatible with the design, even if the diffusivity of the gas was 10-fold lower than that in PDMS. Alternately, if the diffusivity was as high as that in PDMS, the solubility could be more than an order of magnitude lower.

In terms of permeability:

P=DS

The permeability of PDMS is 800 Barrer (1 Barrer=10 cm³(STP)·c/cm²·s·cm Hg) (44).

This model suggests that any membrane with an oxygen permeability >80 Barrer will work with the design, and the permeability could probably be even lower (still relatively high diffusivity, but solubility could be lower).

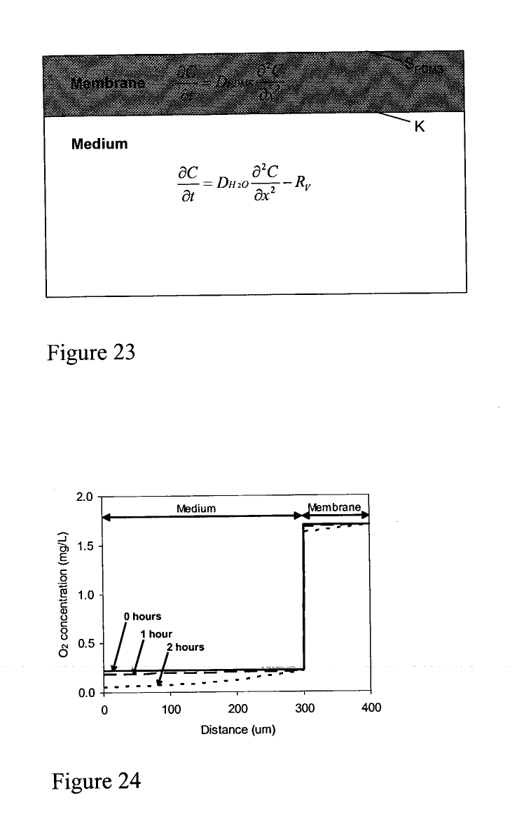

The model described above establishes the feasibility of the microfermentor design based on a steady state analysis. The design of the microfermentor can be further validated by a transient analysis of the oxygen transport during growth. FIG. 23 shows the two oxygen transport regions in the microfermentor (parameters used are listed in Table 4). The transient model assumes exponential growth (the most oxygen demanding growth phase) of homogeneously-dispersed cells, and it is based on the three equations below.

$$\begin{matrix}\frac{\partial C}{\partial t} = D \frac{{\partial }^{2} C}{\partial {x}^{2}} - {R}_{V} \\ {R}_{V} = OxygenUptakeRate = - {Y}_{O / X} \frac{ⅆ N}{ⅆ t} \\ \frac{ⅆ N}{ⅆ t} = N {μ}_{max}\end{matrix}$$

FIG. 24 shows the oxygen concentration profile across the membrane and the microbioreactor at increasing time. As in the previous example, the major resistance to mass transfer occurs in the medium rather than the membrane, a result of the low solubility of oxygen in water. It was found that a depth of 300 μm allowed sufficient oxygenation to reach a final cell number ˜10¹² cells/L. From this figure it is also apparent that a concentration gradient exists within the medium as oxygen is gradually depleted.

TABLE 4
List of parameters used in models

| Parameter | Definition | Value | Reference |
|---|---|---|---|
| S_{PDMS} | ^{†}Solubility of O₂ in PDMS | 0.18 cm³(STP)/ | 44 |
| | | cm³ · atm | |
| D_{PDMS} | ^{†}Diffusivity of O₂ in PDMS | 3.4 × 10⁻⁵ cm²/s | 44 |
| S_{H2O} | ^{†‡}Solubility of O₂ in water | 7.36 mg/ | 45 |
| D_{H2O} | ^{†‡}Diffusivity of O₂ in | 2.5 × 10⁻⁵ cm²/s | 45 |
| | water | | |
| K | ^{†‡}PDMS-H₂O partition | 0.129 | Calculated |
| | coefficient | | |
| Y_{O/X} | Yield of biomass on oxygen | 1 g_{O2} consumed/ | Literature |
| | | g_{DCW} (Dry Cell | |
| | | Weight) produced | |
| N₀ | Initial number of cells | 3.8 × 10⁷ cells/m | Experiment |
| t_{d} | Doubling time | 25 min | Experiment |
| μₘₐₓ | Maximum specific growth | 0.0278 min⁻¹ | Experiment |
| | rate Conversion | 2.8 × 10⁻¹³ g_{Dcw}/ | 82 |
| | | E. coli cell | |
| C* | Percent oxygen at | 100% | Definition |
| | saturation | | |

†At 35° C., in equilibrium with 0.21 atm of oxygen
‡Values for pure water were used since only 8 g/ of glucose was present in the medium
*Critical oxygen concentration = 0.0082 mmol/ (˜3.6% of air saturation) (55)

TABLE 5
List of variables used in models

| Parameter | Description |
|---|---|
| C | Concentration of oxygen |
| D | Diffusivity of O₂ in each phase |
| R_{V} | Volumetric accumulation term |
| N | Number of cells |
| μ | Specific growth rate of cells |

### Example 3

### Setup of a Microscale Bioreactor System

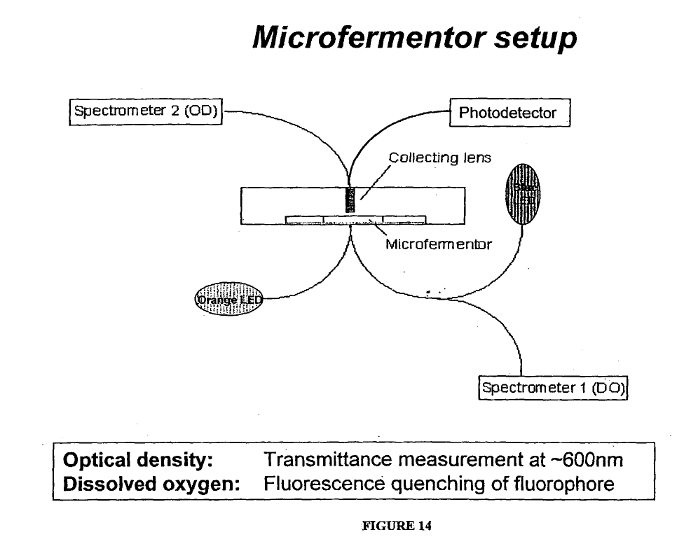

FIG. 14 shows a schematic of a microscale bioreactor system with associated optical excitation and detection sources. Optical fibers transmit light to the bottom of the fermentor. Biomass is monitored by measuring the amount of light transmitted to the collecting lens above.

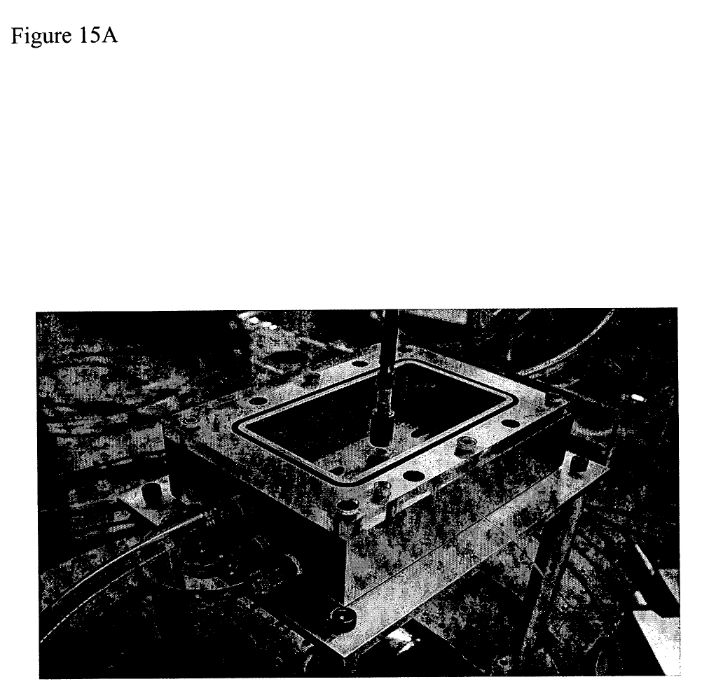

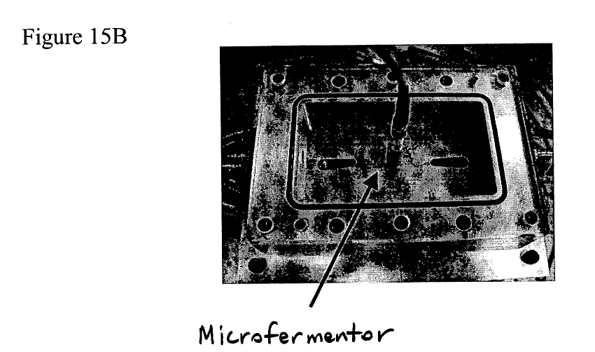

The microfermentor is placed in an enclosed chamber designed to facilitate environmental control during fermentations. The chamber is fabricated from aluminum and has a screw-on lid that can be sealed with an O-ring. FIG. 15A depicts the chamber with the microfermentor inside. FIG. 15B is a second view to more clearly show the microfermentor. (Note that the slide that forms the base of the microfermentor is transparent.) In this system, evaporation from the microfermentor is controlled by making the chamber airtight and by maintaining the air within the chamber at high humidity, e.g., 100% humidity. This is accomplished by placing open reservoirs of water beside the microfermentor within the chamber. The large volume of the chamber (˜190 cm³) as compared to the volume of the microfermentor ensures that sufficient oxygen is present to supply the needs of the growing bacteria throughout a run. Less than 1% of available oxygen is consumed by respiring bacteria during the course of a 12 hour fermentation. The chamber is maintained at a constant, desired temperature by flowing heated water from a water bath through channels within the chamber base using a heating circulator (DC-10, Thermo Haake, Karlsruhe, Germany).

Optical fibers run to the center of the chamber cover and base, above and directly below the microfermentor respectively. These fibers allow both transmissive and reflective optical measurements to be made. The fiber positioned above the microfermentor is attached to a collecting lens (F230SMA-a), ThorLabs) that increases the solid angle of capture of light emitted from the fiber below and transmitted through the microfermentor.

### Example 4

### Monitoring Bioprocess Parameters of Cells Cultured in a Microscale Bioreactor

Preparation and Inoculation of Cells

E. coli were cultured at 37° C. for 12 hours in LB medium+amp with or without addition of glucose (43). Immediately prior to introduction of the cells into the microfermentor, a 5% inoculum was introduced into fresh medium. Prior to inoculation the microfermentor was sterilized by a 60 second exposure to UV light at a wavelength of 254 nim. Inoculation of the cells was accomplished using a syringe to drive fluid through the channels and into the vessel interior. The channel holes, which self-seal to a large extent, were then further sealed using epoxy to minimize evaporation. Various epoxies and adhesives (e.g., Epoxy—ITW Performance Polymers, Part No: 46409/20845, Silicone adhesive—American Sealants, Inc., ASI #502 Silicone) have been used with no evidence of deleterious effects due to contact with cells. However, biocompatibility of the adhesive may be a consideration. Once filled, the microfermentor was placed into the chamber and secured to the base. The chamber was then closed with an airtight seal and optically sealed to prevent stray light from interfering with subsequent measurements.

Measurement of Biomass

Quantification of biomass was based on the transmission of light through the microfermentor. The light source is an orange LED with a peak wavelength of 609 nm or a helium neon (HeNe) laser with a peak wavelength of 636 μm. This light is coupled into a 600 μm optical fiber as described above. A 600 μm fiber above the microfermentor carries the transmitted light to a spectrometer (OCS-PDA, Control Development). A photodetector (PDA55, ThorLabs) is used to check for temporal power drift from the light source.

Optical density (OD) is calculated using:

OD=log₁₀(1/T)

where T=transmittance of light calculated from the intensity, I, using:

T=I_{signal}/I_{ref}

A curve for optical density as measured in a cuvette by a conventional spectrometer was obtained by diluting a sample of the fermentation medium by a factor of 10, so that it fell within into the linear portion of the spectrometer range. This value of the optical density was then used to determine the actual optical density at all other dilutions.

Measurement of Dissolved Oxygen

Fluorescence quenching of Ruthenium II tris(4,7-diphenyl-1,1-phenanthroline)²⁺ was used to measure the dissolved oxygen at the bottom of the microfermentor. The glass slide that forms the base of the microfermentor was coated with sol-gel containing this compound. These slides are available commercially (Foxy sol-gel slides, Ocean Optics). A bifurcated cable carries light at the excitation wavelength to the base of the microfermentor. The light source is USB-LS-450, Ocean Optics). Emitted light that is captured by the optical fiber is then carried back to the spectrometer (USB2000-FL, Ocean Optics), where the percent dissolved oxygen is calculated using OOISensors Software (Ocean Optics).

Results

Typical viable cell counts (based on optical density calculated from transmission data) for E. coli growing in the microfermentor in LB+amp medium without the addition of glucose indicate a cell density of approximately 4×10⁹ cells/mL (4×10¹² cells/L), comparable to that employed in large-scale fermentation processes.

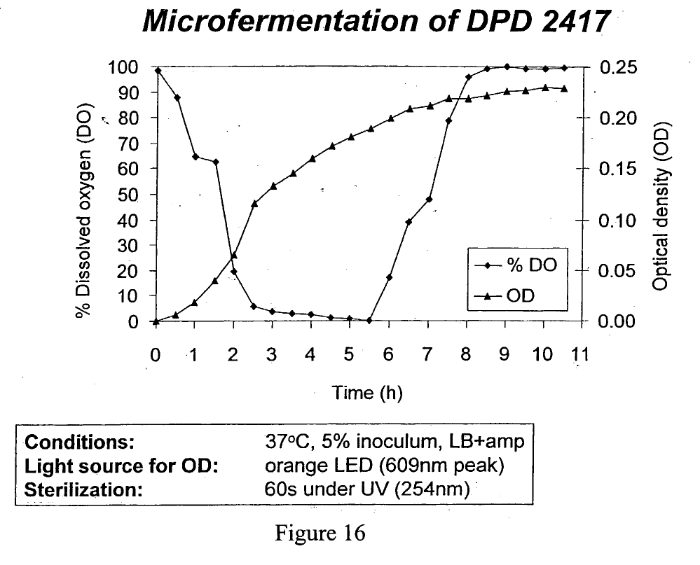

FIG. 16 shows optical density and dissolved oxygen data obtained from batch fermentation of E. coli cultured in LB+amp in a microfermentor. Oxygen was provided via the PDMS membrane, and no active stirring of the medium took place. Dissolved oxygen was measured using the Ru-based oxygen sensor. Three distinct phases of growth can be observed in FIG. 16. During the first stage, bacteria are in the exponential phase of growth and are multiplying with an apparent doubling time of 30 minutes. (The doubling time is referred to as “apparent” because in accordance with the results described above, the optical density predictably underestimates the actual biomass.) During this first stage enough oxygen is supplied by diffusion to support this rapid growth. The second stage is reached when the level of measurable oxygen in the medium drops close to zero, and oxygen is utilized by the bacteria as quickly as it diffuses into the microfermentor vessel. During this phase the bacteria switch to linear growth. Finally, the third stage shows the bacteria reaching a stationary phase. During this stage oxygen levels return to saturation. The time required to reach saturation can be predicted from the non-steady-state one dimensional diffusion equation:

∂C/∂T=D(∂²C/∂x²)

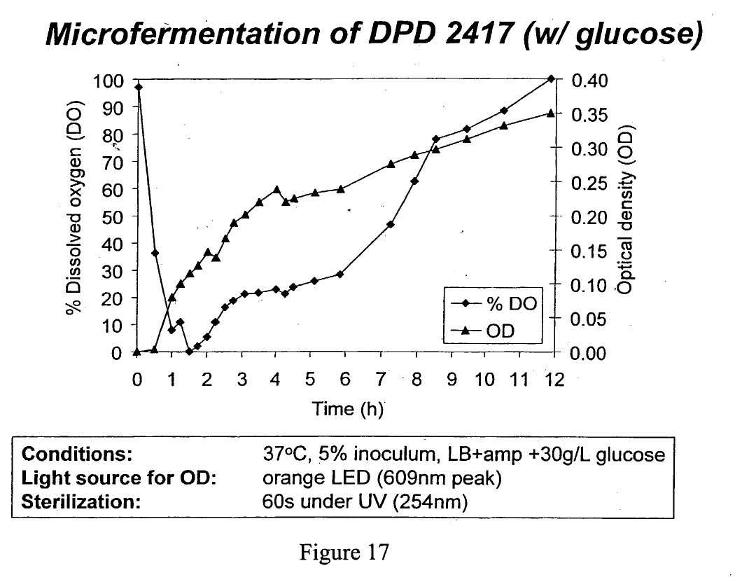

This results in an estimate on the order of minutes needed to fully reoxygenate the microfermentor to a depth of 300 μm. This time is shorter than the measured time of 2.5 hours shown in FIG. 17, but the longer reoxygenation time required is consistent with the observed accompanying increase in biomass. FIG. 17 shows a comparable curve for E. coli cultured in LB/amp+30 g/liter glucose. FIGS. 18A and 18B show fermentation of E. coli cultured in LB/amp+30 g/liter glucose in a 0.5 liter bench scale fermentor (Sixfors) at 37 degrees, 500 RMP, aeration 2 VVM (50% O₂, 50% N₂). The growth curve and curve of oxygen concentration within the microscale bioreactor show similar trends to that obtained in the bench-scale fermentor.

### Example 5

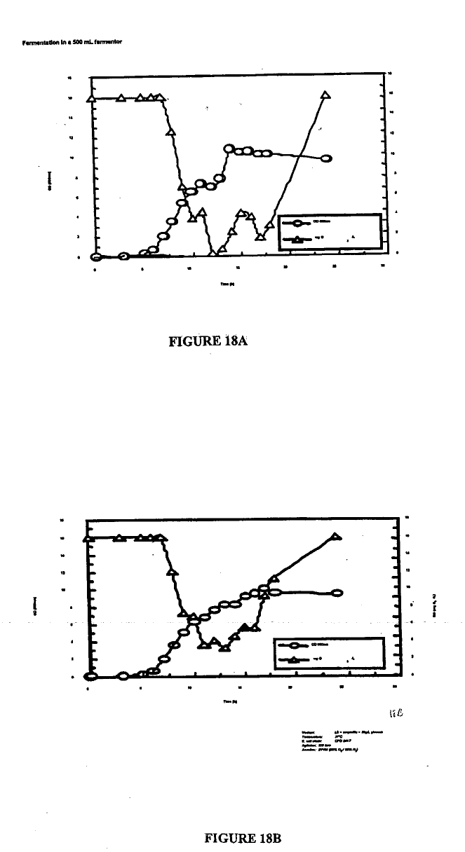

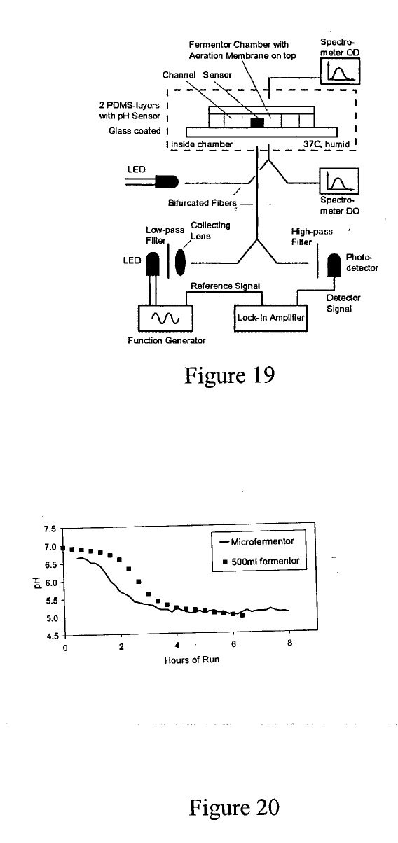

FIG. 19 shows a schematic diagram of an embodiment of the invention in which biomass, dissolved oxygen, and pH can be measured simultaneously. The microfermentor was constructed and housed in a chamber essentially as described in Examples 3 and 4. Optical density was used as a measurement of biomass. To measure dissolved oxygen, the fluorophore described above, whose fluorescence is quenched in the presence of oxygen, was excited by an LED, and the intensity of the emission was read using a spectrometer. The dissolved oxygen can also be measured using a fluorescence lifetime measurement. The pH was measured by detecting fluorescence lifetime changes in a pH sensorfoil (Presens, Regensburg, Germany) located within the microfermentor. The lifetime of the fluorescence was measured by detecting the phase-shift of the fluorescence with respect to the intensity-modulated LED using a lock-in amplifier. Bifurcated optical fibers were inserted into the bottom and top of the chamber to allow the various optical measurements to be performed.

Dissolved oxygen and biomass were measured as described in Example 4, and similar results were obtained. FIG. 20 is a graph comparing pH curves in the microfermentor and in a 0.5 L bench scale fermentor (Sixfors). The pH in the bench-scale fermentor drops after approximately 2 hours and reaches a pH of ˜5 after 6 hours. A similar trend can be observed in the microfermentor, in which the pH drops to ˜5 after 5 hours.

### Example 6

### Strain Selection Using a Microscale Bioreactor Array

Xylitol, a naturally occurring sugar alcohol, is a promising low-calorie sweetener that has lower calories than sucrose and yet exhibits comparable sweetness. It is presently as a dental caries preventive sweetener and also finds use in fluid therapy in the treatment of diabetes. For these reasons, it is expected that the demand of xylitol will increase in future. Thus the demand for xylitol is expected to increase in future.

Current industrial production of xylitol mainly relies on hydrogenation of D-xylose as disclosed in U.S. Pat. No. 4,008,285. D-Xylose used as a raw material is obtained by hydrolysis of plant materials such as trees, straws, corn cobs, oat hulls and other xylan-rich materials. However, such D-xylose, which is produced by hydrolysis of plant materials, is rather expensive and has low purity. Other production methods, utilizing D-arabitol as a starting material, are complex and involve multiple steps. Attempts to use genetic engineering to develop a microorganism with improved ability to produce xylitol have met with only limited success. Therefore, it is desirable to identify a microorganism that can produce xylitol through a single step by fermentation starting from glucose as used in the production of other saccharides and sugar alcohols.

To address this need, osmophilic microorganisms are collected from nature by enrichment culture. A medium containing 20% D-glucose, 1% yeast extract (Difco), and 0.1% urea is introduced into test tubes in an amount of 4 ml each, and sterilized at 120° C. for 20 minutes. Soil samples collected from various locations in the Cambridge, Mass. area are inoculated into the medium, and cultured at 30° C. for 4 to 7 days with shaking. When bacterial growth is observed, the cultures are plated on an agar plate having the same composition, and incubated at 30° C. for 1 to 3 days. Single colonies were isolated.

Approximately 2000 strains of osmophilic bacteria obtained as described above are cultured in individual microfermentors within a microfermentor array in a medium containing 20% (w/v) D-glucose, 0.1% urea, and 0.5% yeast extract at 30° C. for periods ranging from 12 hours to 5 days. The microfermentors have a working volume of 5 μl and are equipped with means to monitor biomass and oxygen concentration. Each microfermentor delivers oxygen to the interior of the microfermentor vessel via a PDMS aeration membrane. Each strain is introduced into 18 individual microfermentors using access channels. This allows 3 cultures to be terminated at each of 6 time points for each strain. The microfermentor array is maintained in a chamber as described in Example 3, which controls temperature and humidity. Biomass and dissolved oxygen concentration are monitored during the culture period, and data is accumulated using an appropriate software program. After an appropriate culture period (12, 24, 48, 72, 96, or 120 hours), all medium is removed from each microfermentor to be terminated at that time point and analyzed by HPLC to screen for a strain having the ability to produce xylitol.

### Example 7

### Strain Characterization and Process Parameter Optimization Using a Microscale Bioreactor Array

(1) Measurement of Acid Production and Cell Growth with Various Carbon Sources

Xylitol producing strains identified as in Example 6 are each cultured in individual microfermentors in a medium containing one of various carbon sources (1%), and presence of formed acid is determined. The following carbon sources are tested: xylose, arabinose, glucose, galactose, mannose, fructose, sorbase, sucrose, maltose, rhamnose, glycerol, mannitol, sorbitol, lactose, starch, and ethanol. The strains are pre-cultured in flasks in YPG medium at 28° C. for one day and then washed with 0.5% yeast extract solution. Since 5 strains and 16 carbon sources are tested, there is a total of 80 combinations.

Thirty microfermentors in a microfermentor array are inoculated with cells in YPC medium for each strain/carbon source combination, making a total of 2400 microfermentors. This allows 10 cultures to be terminated at each of 3 time points for each strain. (YPC is medium containing 0.5% yeast extract (Difco), and 1% of one of the various carbon sources sterilized by heating at 120° C. for 20 minutes prior to addition of the sterile carbon source. Depending on the particular pH sensor, the medium may contain a pH-sensitive dye such as bromocresol purple. The microfermentors have a working volume of 5 μl and are equipped with means to optically monitor biomass, oxygen concentration, and pH. Each microfermentor delivers oxygen to the interior of the microfermentor vessel via a PDMS aeration membrane.

The microfermentor array is maintained in a chamber as described in Example 3, which controls temperature and humidity. Biomass, dissolved oxygen concentration, and pH are monitored during the culture period, and data is accumulated using an appropriate software program. Cultures are maintained at 28° C. for 4, 5, or 6 days. After an appropriate culture period, all medium is removed from each microfermentor to be terminated at that time point and analyzed by HPLC to determine the amount of xylitol produced. The data can be used to select an appropriate strain and culture medium for a production scale fermentation process for the production of xylitol.

(2) Effect of NaCl, Acetic acid or Ethanol Addition on Growth

Xylitol producing strains identified as in Example 6 are each cultured in individual microfermentors in YPM medium containing NaCl, ethanol, and/or acetic acid at a range of concentrations to determine the effect of these additives, singly or in combination, on growth. The xylitol producing strains and Acetobacter aceti strain NCIB 8621 as a control are pre-incubated in YPG medium (1% yeast extract (Difco), 1% peptone, sterilized by heating at 120° C. for 20 minutes, followed by addition of D-glucose to 7%) at 28° C. for one day, washed, and resuspended into medium with the one or more of the various additives at a range of concentrations. For each additive, 5 different concentrations are tested.

Thirty microfermentors are inoculated for each additive/concentration combination, allowing identical 10 cultures to be terminated at each of 3 time points. The microfermentors have a working volume of 5 μl and are equipped with means to optically monitor biomass, oxygen concentration, and pH. Each microfermentor delivers oxygen to the interior of the microfermentor vessel via a PDMS aeration membrane. The microfermentors are maintained in a chamber as described in Example 3, which controls temperature and humidity. Biomass, dissolved oxygen concentration, and pH are monitored during the culture period, and data is accumulated using an appropriate software program. Cultures are maintained at 28° C. for 4, 5, or 6 days. After an appropriate culture period, all medium is removed from each microfermentor to be terminated at that time point and analyzed by HPLC to determine the amount of xylitol produced. The data can be used to select an optimum strain and culture medium for a production scale fermentation process for the production of xylitol.

### REFERENCES

1. D. M. Disley, P. R. Morrill, K. Sproule, C. R. Lowe, “An optical biosensor for monitoring recombinant proteins in process media,” 14, 481-493 (1999).

2. D. E. Cane, C. T. Walsh, C. Khosla, “Harnessing the biosynthetic code: combinations, permutations, and mutations,” Science 282, 63-68 (1998).

3. M. Kleerebezemab, P. Hols, J. Hugenholtz, “Lactic acid bacteria as a cell factory: rerouting of carbon metabolism in Lactococcus lactis by metabolic engineering,” Enzyme Microb Technol. 26, 840-848 (2000).

4. D. C. Cameron, N. E. Altaras, M. L. Hoffman, A. J. Shaw, “Metabolic engineering of propanediol pathways,” Biotechnol Prog. 14, 116-25 (1998).

5. D. H. Pieper, W. Reineke, “Engineering bacteria for bioremediation,” Curr. Opin. Biotechnol. 11 262-70 (2000).

6. J. Ohlrogge, “Plant metabolic engineering: are we ready for phase two?,” Curr. Opin. Plant Biol. 2, 121-122.

7. S. Guillouet, A. A. Rodal, G.-H. An, P. A. Lessard, and A. J. Sinskey “Expression of the Escherichia coli catabolic threonine dehydratase, in Corynebacterium glutamicum and its effect on isoleucine production,” Applied and Environmental Microbiology 6, 3100-3107 (1999).

8. M. W Losey, M. A., Schmidt, and K. F. Jensen, “A micro packed-bed reactor for chemical synthesis”, In Microreacttion Technology: Industrial Prospects (Ed, Ehrfeld, W.) Springer, Berlin, pp. 277-286 (2000).

9. R. Srinivasan, I.-M. Hsing, P. E. Berger, K. F. Jensen, S. L Firebaugh, M. A. Schmidt, M. P Harold, J. J. Lerou, and J. F Ryley. “Micromachined reactors for catalytic partial oxidation reactions”. AIChE Journal, 43, 3059-3069 (1997).

10. T. M Floyd, M. W. Losey, S. L. Firebaugh, K. F. Jensen and M. A Schmidt. “Novel liquid phase microreactors for safe production of hazardous specialty chemicals,” In Microreaction Technology: Industrial Prospects (Ed. Ehrfeld, W.) Springer, Berlin, pp. 171-180 (2000).

11. S. L Firebaugh, K. F. Jensen and M. A. Schmidt, “Miniaturization and integration of photoacoustic detection with a microfabricated chemical reactor system,” In Micro Total Analysis Systems 2000 (Eds, Berg, A. v. d., Olthuis, W. and Berveld, P.) Kluwer Academic Publishers, Dordrecht, pp. 49-52 (2000).

12. M. W Losey, M. A., Schmidt, and K. F. Jensen, “Microfabricated multiphase packed-bed reactors: characterization of mass transfer and reactions,” I&EC Research submitted (2000).

13. J. J Jackmann, T. Floyd, M. A. Schmidt, and K. F. Jensen, “Development of methods for on-line chemical detection with liquid-phase microchemical reactors using conventional and unconventional techniques,” In Micrototal Analysis Systems 2000 (Eds, Berg, A. v. d., Olthuis, W. and Bergveld, P.) Kluwer Academic Publishers, Dordrecht, pp. 155-159 (2000).

14. S. W. Lee, P. E. Laibinis, “Protein resistant coatings for glass and metal oxide surfaces derived from oligo(ethylene glycol)-terminated alkyltrichlorosilanes.” Biomaterials 19, 1669-1675 (1998).

16. N. Y. Kim, P. E. Laibinis, “Covalent modification of hydrogen-terminated silicon surfaces,” In New Directions in Materials Synthesis, C. H. Winter, Ed.; ACS Symposium Series, 727, 157-168 (1999).

17. Y. K. Namyong, N. L. Jeon, I. S. Choi, S. Takami, Y. Harada, K. R, Finnie, G. S. Girolami, R. G. Nuzzo, G. M. Whitesides, and P. E. Laibinis, “Surface-initiated ring-opening metathesis polymerization on silicon,” Macromolecules, 33, 2793-2795 (2000).

18. J. J. Hickman, D. Ofer, C. Zou, M. S. Wrighton, P. E. Laibinis, G. M. Whitesides, “Selective functionalization of gold microstructures with ferrocenyl derivatives via reaction with thiols or disulfides: Characterization by electro-chemistry and auger electron spectroscopy,” J. Am. Chem. Soc., 113, 1128-1132 (1991).

19. J. J. Hickman, D. Ofer, M. S. Wrighton, P. E. Laibinis, G. M. Whitesides, “Molecular self-assembly of two-terminal, voltammetric microsensors with an internal reference,” Science, 252, 688-691 (1991).

20. R. Michalitsch; P. E. Laibinis, “Electrochemical halide detection by gold electrodes exposing a layer of silver atoms,” Angewandte Chemie (submitted).

21. Q.-Y. Li, L. M. Davis, “Rapid and efficient detection of single chromophore molecules in aqueous solution,” Applied Optics, 34, 3208-3217 (1995).

22. A. G. Mignani, F. Baldini, “In-vivo biomedical monitoring by fiber-optic systems,” J. of Lightwave Tech., 13, 1396-1406 (1995).

23. I.-Y. Li, L. M. Davis, Applied Optics, 34, 3208-3217 (1995)

24. C.-M. Chun, W. Lo, K.-Y. Wong, “Application of a luminescence-based pH optrode to monitoring of fermentation by Klebsiella pneumoniae,” Biosensors and Bioactuators, 15,7-11 (2000).

25. M. E. Lippitsch, S. Draxler, D. Kieslinger, Sensors and Actuators B, 38-39, 96-102 (1997).

26. S. Draxler, M. E. Lippitsch, “pH sensors using fluorescence decay time,” Sensors and Acutators B, 29, 199-203 (1995).

27. B. J. Feilmeier, G. Iseminger, D. Schroeder, H. Webber, G. J. Phillips, “Green fluorescent protein functions as a reporter for protein localization in escherichia coli,” J. Bacteriol., 182, 4068-4076 (2000).

28. D. H. Edwards, H. B. Thomaides, J. Errington “Promiscuous targeting of bacillus subtilis cell division protein DivIVA to division sites in Escherichia coli and fission yeast,” Embo. J, 19, 2719-27 (2000).

29. D. Aldon, B. Brito, C. Boucher, S. Genin, “A bacterial sensor of plant cell contact controls the transcriptional induction of Ralstonia solanacearum pathogenicity genes,” Embo J. 19, 2304-14 (2000).

30. K. R. Finer, J. J. Finer “Use of Agrobacterium expressing green fluorescent protein to evaluate colonization of sonication-assisted Agrobacterium-mediated transformationtreated soybean cotyledons” Lett Appl Microbiol. 30, 406-410 (2000).

31. M. F. Garcia-Parajo, G. M. Segers-Nolten, J. Veerman, J. Greve, N. F. van Hulst “Real-time light-driven dynamics of the fluorescence emission in single green fluorescent protein molecules,” Proc Natl Acad Sci U.S.A., 97, 7237-7242 (2000).

32. A. K. Dunn, J. Handelsman, “A vector for promoter trapping in Bacillus cereus,” Gene, 226, 297-305 (1999).

33. J. N. Demas and B. A. DeGraff, “Design and Applications of Highly Luminescent Transition Metal Complexes”, Analytical Chemistry, vol. 63, ppl 829-837, 1991.

34. S. B. Bamobt, et al., “Phase Fluorometric Sterilizable Optical Oxygen Sensor”, Biotechnology and Bioengineering, vol. 43, ppl 1139-1145, 1994.

35. A. K. McEvoy, et al., “Dissolved oxygen sensor based on fluorescence quenching of oxygen-sensitive ruthenium complexes immobilized in sol-gel derived porous silicon coatings”, Analyst, vol. 121, pp. 785-788, 1996.

36. Z. Zhujun, et al., “A Carbon Dioxide sensor Based on Fluorescence”, Analytica Chimica Acta, 160: 305-309 (1984).

37. Y. Kawabata, et al., “Fiber-Optic Sensor for Carbon Dioxide with a pH Indicator Dispersed in a Poly (Ethylene Glycol) Membrane”, Analytica Chimica Acta, 219:223-229 (1989).

38. B. Weigl, et al., “Chemically and mechanically resistant carbon dioxide optrode based on a covalently immobilized pH indicator”, Analytica Chimica Acta, 282:335-343 (1993).

39. Y. Kostov, et al., “Low-cost Microbioreactor for High Throughput Bioprocessing”, Biotechnology and Bioengineering, vol. 72, pp. 346-352, 2001.

40. S. DeWitt, “Microreactors for Chemical Synthesis”, Curr. Op. in Biotechnology, 3:350-356, 1999.

41. F. Neidhardt, (ed.), “Escherichia Coli and Salmonella: Cellular and Molecular Biology, CD-ROM edition, September 1999, Amer Society for Microbiology; ISBN: 1555811647.

42. E. Winzeler, “Functional characterization of the S. cerevisiae genome by gene deletion and parallel analysis”, Science Aug. 6, 1999;285(5429):901-6.

43. J. Sambrook, et al., Molecular Cloning—A Laboratory Manual, 2^{nd} ed.: Cold Spring Harbor Laboratory Press, 1989.

44. T. C. Merkel, et al., “Gas sorption, diffusion, and permeation in poly(dimethylsiloxane)”, J. of Polymer Science, Part B-Polymer Physics, vol. 38, ppl 415-434, 2000.

45. Perry's Chemical Engineering Handbook: R. R. Donnelley & Sons Company, 1984.

46. B. Atkinson and F. Mavituva, Biochemical Engineering and Biotechnology Handbook. New York: The Nature Press, 1983.

47. Rooney, M. T. V. and W. R. Seitz, An optically sensitive membrane for pH based on swellable polymer microspheres in a hydrogel. Analytical Communications, 1999. 36(7): p. 267-270.

48. Vacik, J., et al., The Effect of Ph and Temperature on the Electrical-Conductivity of Membranes Made of Methacrylic-Acid Co-Polymers. Collection of Czechoslovak Chemical Communications, 1983. 48(11): p. 3071-3078.

49. Vacik, J. and J. Kopecek, Specific Resistances of Hydrophilic Membranes Containing Ionogenic Groups. Journal of Applied Polymer Science, 1975. 19(11): p. 3029-3044.

50. Sheppard, N. F., R. C. Tucker, and S. Salehihad, Design of a Conductimetric Ph Microsensor Based on Reversibly Swelling Hydrogels. Sensors and Actuators B-Chemical, 1993. 10(2): p. 73-77.

51. Sheppard, N. F., M. J. Lesho, and P. McNally, Microfabricated Conductimetric Ph Sensor. Sensors and Actuators B-Chemical, 1995. 28(2): p. 95-102.

52. Zabriskie, D, et al. “Estimation of fermentation biomass concentration by measuring culture fluorescence”, Appl. Eur. Microbiol. 1978, Vol. 35(2), pp. 337-343;

53. Marose, S., et al. “Two-dimensional fluorescence spectroscopy: A new tool for online bioprocess monitoring”, Biotechnology Progress, 1998, 14, pp. 63-74).

54. Stephanopolous, G., ed. Bioprocessing. Second ed. Biotechnology, ed. H.-J. Rehm, et al. Vol. 3. 1993, VCH Publishers Inc.: New York.

55. Bailey, J. E. and D. F. Ollis, Biochemical Engineering Fundamentals. Second ed. McGraw-Hill chemical engineering series. 1986: McGraw-Hill, Inc.

56. Demas, J., et al., “Applications of luminescent transition platinum group metal complexes to sensor technology and molecular probes”, Coordination Chemistry Reviews, 211, 2001, pp. 317-351.

57. Demas, J., et al. “Oxygen sensors based on luminescence quenching”, Analytical Chemistry News and Features, Dec. 1, 1999, pp. 793A-800A.

58. Duffy, D., et al., “Rapid prototyping of microfluidic systems in poly(dimethylsiloxane),” Analytical Chemistry, vol. 70, pp. 4974-4984, 1998.

59. Mulder, M., Basic Principles of Membrane Technology. Second ed. 1996: Kluwer Academic Publishers.

60. Tamai, Y., H. Tanaka, and K. Nakanishi, Molecular Simulation of Permeation of Small Penetrants through Membranes .1. Diffusion-Coefficients. Macromolecules, 1994. 27(16): p. 4498-4508.

61. Tamai, Y., H. Tanaka, and K. Nakanishi, Molecular Simulation of Permeation of Small Penetrants through Membranes .2. Solubilities. Macromolecules, 1995. 28(7): p. 2544-2554.

62. Watson, J. M. and M. G. Baron, The behaviour of water in poly(dimethylsiloxane). Journal of Membrane Science, 1996. 110(1): p. 47-57.

63. Fritz, L. and D. Hofmann, Molecular dynamics simulations of the transport of water-ethanol mixtures through polydimethylsiloxane membranes. Polymer, 1997. 38(5): p. 1035-1045.

64. Merkel, T. C., et al., Gas sorption, diffusion, and permeation in poly(dimethylsiloxane) Journal of Polymer Science Part B-Polymer Physics, 2000. 38(3):

p. 415-434.

65. Stern, S. A., V. M. Shah, and B. J. Hardy, Structure-Permeability Relationships in Silicone Polymers. Journal of Polymer Science Part B-Polymer Physics, 1987. 25(6): p. 1263-1298.

66. Pinnau, I. and L. G. Toy, Gas and vapor transport properties of amorphous perfluorinated copolymer membranes based on 2,2-bistrifluoromethyl-4,5-difluoro-1,3-dioxole/tetrafluoroethylene. Journal of Membrane Science, 1996. 109(1): p. 125-133.

67. C. McDonagh, et al., “Tailoring of sol-gel films for optical sensing of oxygen in gas and aqueous phase”, Analytical Chemistry, Vol. 70(1), Jan. 1, 1998, pp. 45-50.

68. S. Parekh, et al., “Improvement of microbial strains and fermentation processes”, Appl. Microbiol. Technol., 54:287-301, 2000.

69. A. L. Demain and J. E. Davis, Manual of industrial microbiology and technology, 2^{nd} ed., Am. Soc. Microbiol. Press, Washington, D.C., 1998.

70. Swarz, J. R., “Advances in Escherichia coli production of therapeutic proteins”, Current Opinion in Biotechnology, 2001. 12: p. 195-201.

71. Baneyx, F., “Recombinant protein expression in Escherichia coli”, Current Opinion in Biotechnology, 1999. 10(5): p. 411-421.

72. Cereghino G P, Cregg J M., “Applications of yeast in biotechnology: protein production and genetic analysis”, Curr Opin Biotechnol. October 1999;10(5):422-7.

73. S. Hashimoto and A. Ozaki, “Whole microbial cell processes for manufacturing amino acids, vitamins or ribonucleotides”, Curr Opin Biotechnol. December 1999;10(6):604-8.

74. Tanaka, H., J. Takahashi, and K. Ueda, “Studies on Effect of Agitation on Mycelia in Submerged Mold Culture 0.3. Standard for Intensity of Agitation Shock on Mycelia on Agitation of Mycelial Suspensions”, Journal of Fermentation Technology, 1975. 53(1): p. 18-26.

75. Oldshue, Y., “Fermentation mixing scale-up techniques”, Biotechnology and Bioengineering, 1966. 8: p. 3-24.

76. Hauser, H., et al. (eds.), Mammalian Cell Biotechnology in Protein Production, W de Gruyter, 1997.

77. Campbell, S., The Science and Engineering of Microelectronic Fabrication, 2nd Ed., Oxford: 2001.

78. Madou, M., Fundamentals of Microfabrication, Boca Raton: CRC Press, 1997.

79. Lahann, J., “Reactive Polymer Coatings: A Platform for Patterning Proteins and Mammalian Cells onto a Broad Range of Materials”, Langmuir, May 2002.

80. Ferraro, J. R., et al., Introductory Raman Spectroscopy, Academic Press; 2nd edition (Oct. 28, 2002).

81. Laserna, J. J. (Ed.) Modern Techniques in Raman Spectroscopy, John Wiley & Son Ltd; 1 edition (Aug. 28, 1996).

82. Pramanik, J. & Keasling, J. D. Stoichiometric model of Escherichia coli metabolism: incorporation of growth-rate dependent biomass composition and mechanistic energy requirements. Biotechnology and Bioengineering 56, 398-421 (1997).

1. A microscale bioreactor comprising:
a vessel having an interior volume of less than 200 microliters; and
means for providing oxygen to the vessel at a concentration sufficient to support cell growth.

2. The microscale bioreactor of claim 1, further comprising at least one channel extending from and in communication with the vessel.

3. The microscale bioreactor of claim 2, further comprising:
means for introducing a component into the vessel or removing a sample from the vessel via a channel.

4. The microscale bioreactor of claim 1, wherein the interior volume is between approximately 100 and 200 microliters, inclusive.

5. The microscale bioreactor of claim 1, wherein the interior volume is between approximately 50 and 100 microliters, inclusive.

6. The microscale bioreactor of claim 1, wherein the interior volume is between approximately 5 and 50 microliters, inclusive.

7. The microscale bioreactor of claim 1, wherein the interior volume is approximately 5 microliters.

8. The microscale bioreactor of claim 1, wherein the means for providing oxygen is integrated into a vessel wall.

9. The microscale bioreactor of claim 1, wherein the means for providing oxygen forms a structural component of the bioreactor.

10. The microscale bioreactor of claim 1, wherein the means for providing oxygen comprises an aeration membrane, and wherein oxygen diffuses through the membrane into the vessel.

11. The microscale bioreactor of claim 10, wherein the membrane comprises a material selected from the group consisting of fluoropolymers and silicones.

12. The microscale bioreactor of claim 10, wherein the membrane comprises polydimethylsiloxane or Teflon AF 2400.

13. The microscale bioreactor of claim 10, wherein the membrane has a permeability of approximately 800 Barrer.

14. The microscale bioreactor of claim 10, wherein the membrane has a permeability of between approximately 600 and approximately 800 Barrer.

15. The microscale bioreactor of claim 10, wherein the membrane has a permeability of between approximately 400 and approximately 600 Barrer.

16. The microscale bioreactor of claim 10, wherein the membrane has a permeability of between approximately 200 and approximately 400 Barrer.

17. The microscale bioreactor of claim 10, wherein the membrane has a permeability of between approximately 80 and approximately 200 Barrer.

18. The microscale bioreactor of claim 10, wherein the membrane is biocompatible.

19. The microscale bioreactor of claim 10, wherein the membrane is optically transparent.

20. The microscale bioreactor of claim 1, wherein the bioreactor supports cell growth for a period of at least approximately 6 hours.

21. The microscale bioreactor of claim 1, wherein the bioreactor supports cell growth for a period of at least approximately 10 hours.

22. The microscale bioreactor of claim 1, wherein the bioreactor supports exponential cell growth for a period of at least approximately 2.5 hours.

23. The microscale bioreactor of claim 1, wherein the bioreactor supports cell growth that achieves a viable cell density of at least 10⁹ cells per liter.

24. The microscale bioreactor of claim 1, wherein the bioreactor supports cell growth that achieves a viable cell density of at least 10¹⁰ cells per liter.

25. The microscale bioreactor of claim 1, wherein the bioreactor supports cell growth that achieves a viable cell density of at least 10¹¹ cells per liter.

26. The microscale bioreactor of claim 1, wherein the bioreactor supports cell growth that achieves a viable cell density of at least 10¹² cells per liter.

27. The microscale bioreactor of any of claims 20 through 26, wherein the cell growth is bacterial cell growth.

28. The microscale bioreactor of claim 1, wherein at least one interior surface of the vessel or of a channel extending from or in communication with the vessel is coated with a substance that alters adsorption of cells.

29. The microscale bioreactor of claim 28, wherein the substance decreases adsorption of cells.

30. The microscale bioreactor of claim 28, wherein the substance increases adherence of cells.

31. The microscale bioreactor of claim 28 wherein the substance is a silanecontaining film.

32. The microscale bioreactor of claim 28, wherein the surface is modified using a Grignard reagent.

33. The microscale bioreactor of claim 28, wherein the surface is modified using a ring-opening metathesis polymerization reaction to form a film.

34. The microscale bioreactor of claim 28, wherein the substance is a polymer.

35. The microscale bioreactor of claim 34, wherein the polymer is a comb polymer comprising a backbone and a plurality of polymeric side chains attached thereto.

36. The microscale bioreactor of claim 34, wherein the backbone is selected to adsorb to a substrate.

37. The microscale bioreactor of claim 34, wherein the polymeric side chains are selected to retard adsorption of proteins, cells, or both.

38. The microscale bioreactor of claim 34, wherein the polymer comprises a poly(acrylic acid) backbone.

39. The microscale bioreactor of claim 34, wherein the polymer comprises poly(ethylene glycol).

40. The microscale bioreactor of claim 1, further comprising:
means for quantification of biomass within the vessel.

41. The microscale bioreactor of claim 40, wherein the means for quantification of biomass comprises optical detection means.

42. The microscale bioreactor of claim 40, wherein the means for quantification of biomass includes a light source and an optical fiber.

43. The microscale bioreactor of claim 1 or claim 40, further comprising:
means for measuring dissolved oxygen within the vessel.

44. The microscale bioreactor of claim 43, wherein the means for measuring dissolved oxygen comprises an optical sensor.

45. The microscale bioreactor of claim 44, wherein the optical sensor comprises a compound whose fluorescence or luminescence varies depending on oxygen concentration.

46. The microscale bioreactor of claim 45, wherein the compound is a ruthenium compound.

47. The microscale bioreactor of claim 45, wherein the compound is Ruthenium II tris(4,7-diphenyl-1,1-phenanthroline)²⁺.

48. The microscale bioreactor of claim 1, further comprising:
means for quantification of biomass within the vessel;
means for measuring dissolved oxygen within the vessel; and
means for measuring at least one other parameter within the vessel.

49. The microscale bioreactor of claim 48, wherein the at least one other parameter is selected from the group consisting of: temperature, pH, carbon dioxide concentration, carbon source concentration, concentration of an ionic species, and concentration of a cellular metabolite.

50. The microscale bioreactor of claim 49, wherein the at least one other parameter is pH.

51. The microscale bioreactor of claim 48, wherein at least one of the means comprises an optical chemical sensor.

52. The microscale bioreactor of claim 1, further comprising:
at least one waveguide sensor.

53. The microscale bioreactor of claim 1, further comprising a self-assembling sensor.

54. The microscale bioreactor of claim 53, wherein the self-assembling sensor comprises an electroactive thiol reagent.

55. The microscale bioreactor of claim 1, further comprising:
means for controlling the temperature within the vessel.

56. The microscale bioreactor of claim 55, wherein the means for controlling the temperature within the vessel comprises a resistance heater.

57. The microscale bioreactor of claim 1, further comprising:
means for controlling the pH of medium within the vessel.

58. The microscale bioreactor of claim 1, further comprising:
means for delivering nutrients to the vessel.

59. A microscale bioreactor comprising:
at least one waveguide sensor.

60. The microscale bioreactor of claim 59, wherein the waveguide sensor incorporates a photodetector.

61. The microscale bioreactor of claim 60, wherein the photodetector comprises a single-photon avalanche diode.

62. A bioreactor system comprising:
the microscale bioreactor of claim 1; and
a chamber sufficiently large to accommodate the microscale bioreactor, wherein the chamber provides means to control at least one environmental parameter.

63. The microscale bioreactor system of claim 62, wherein the chamber controls either temperature or humidity or both experienced by the microscale bioreactor.

64. The microscale bioreactor system of claim 62, further comprising an optical excitation source positioned so as to direct optical excitation into the bioreactor and an optical detection means positioned so as to sense light transmitted by or emitted from the bioreactor.

65. The microscale bioreactor of claim 64, wherein the optical detection means comprises a Raman spectrometer.

66. The microscale bioreactor system of claim 64, wherein the optical excitation source, the optical detection means, or both include an optical fiber.

67. A bioreactor assembly for performing multiple fermentations in parallel comprising:
a plurality of microscale bioreactors as described in any of claims 1, 40, 52, or 55.

68. A bioreactor assembly for performing multiple fermentations in parallel comprising:
a plurality of microscale bioreactors as described in claim 43.

69. A microfermenter system comprising:
one or more microscale bioreactors as described in claim 1, or one or more arrays of such microscale bioreactors, optionally with associated microfluidic components, and one or more of the following: a plate or platform on or in which one more microscale bioreactors or microscale bioreactor arrays, optionally with associated microfluidics, is mounted or housed; a chamber in which the microfermentors or microfermentor arrays, plates, or platforms are enclosed; a pump; sensing means; detection means; energy delivery means; excitation means; analytical equipment; robotics; software; and computers.

70. A microscale bioreactor comprising
first vessel having an interior volume of 1 ml or less for culturing cells; and
a second vessel separated from the first vessel at least in part by a membrane permeable to oxygen and carbon dioxide.

71. The microscale bioreactor of claim 70, wherein the membrane is permeable to cell products and nutrients but not permeable to cells.

72. The microscale bioreactor of claim 70, further comprising:
means for flowing a liquid or gas through the second vessel.

73. A method of selecting a strain that produces a desired product or degrades an unwanted compound comprising steps of:
culturing a plurality of different strains, each in an individual microscale bioreactor as provided in any of claims 1, 40, 52, or 55;
measuring the amount of the desired or unwanted product in each of the microscale bioreactors; and
selecting a strain that produces an optimum amount of a desired product or degrades a maximum amount of the unwanted compound.

74. A method of selecting a bioprocess parameter comprising steps of:
culturing an organism type in a plurality of microscale bioreactors as provided in any of claims 1, 40, 52, or 55, wherein the microscale bioreactors are operated under conditions in which the value of the bioprocess parameter varies and wherein the organism produces a product or degrades a compound;
monitoring biomass in each of the microscale bioreactors; and
identifying the value of the bioprocess parameter that results in optimum biomass, optimum product formation, or optimum compound degradation.

75. The method of claim 74, in which the bioprocess parameter is actively controlled.

76. The method of claim 74, further comprising monitoring at least one bioprocess parameter in addition to biomass.

77. A method of performing a fermentation comprising:
selecting a cell strain in accordance with the method of claim 73; and
culturing the cell strain in a production scale fermentor.

78. A method of performing a fermentation comprising:
culturing cells in a production scale fermentor, wherein one or more bioprocess parameters for the production scale fermentor is selected according to the method of claim 74.
